(19) United States
(12) Patent Application Publication
(10) Pub. No.: US 2025/0114250 A1
(43) Pub. Date: Apr. 10, 2025

(54) GARMENT-LIKE NONWOVEN LAMINATES

(71) Applicant: The Procter & Gamble Company, Cincinnati, OH (US)

(72) Inventors: Kelyn Anne ARORA, Cincinnati, OH (US); J Michael BILLS, Mason, OH (US); Nathan Ray WHITELY, Liberty Twp, OH (US); Kevin Ronald KANYA, Liberty Twp, OH (US); Janet NETON, West Chester, OH (US)

(21) Appl. No.: 18/903,406

(22) Filed: Oct. 1, 2024

Related U.S. Application Data
(60) Provisional application No. 63/656,772, filed on Jun. 6, 2024.
(60) Provisional application No. 63/588,313, filed on Oct. 6, 2023.

(51) Int. Cl. A61F 13/15; A61F 13/00; A61F 13/514

(52) U.S. Cl. CPC A61F 13/15203; A61F 13/00059; A61F 13/51478; A61F 2013/15325

(57) ABSTRACT

An absorbent article having a garment-facing surface, a wearer-facing surface, a topsheet, a backsheet, an absorbent core positioned at least partially intermediate the topsheet and backsheet, and a component comprising a laminate. The laminate includes a first nonwoven substrate, a second nonwoven substrate, and a plurality of elastic strands positioned at least partially intermediate the first and second nonwoven substrate. The first nonwoven substrate includes a first plurality of fibers having a first color and a second plurality of fibers having a second color. The first and second colors are different. The laminate includes a first side and a second side opposite the first side. The first nonwoven substrate forms a portion of the first side and the first side includes a plurality of disruptive coloring clusters. At least a pair of disruptive coloring clusters have a E of greater than two in a relaxed and/or extended state.

### CROSS-REFERENCE TO RELATED APPLICATIONS

This application claims the benefit of U.S. Provisional Application No. 63/588,313, filed Oct. 6, 2023, and U.S. Provisional Application No. 63/656,772, filed Jun. 6, 2024, which claims the benefit of U.S. Provisional Application No. 63/588,313, filed Oct. 6, 2023, which are incorporated by reference herein in their entireties.

### FIELD

The present disclosure is generally directed to garment-like nonwoven laminates, and more particularly, to absorbent articles comprising garment-like nonwoven laminates having a disruptive coloring effect.

### BACKGROUND

Absorbent articles are used to absorb and contain bodily exudates (e.g., urine, menses, and BM) in infants, children, and adults. Absorbent articles may comprise diapers, pants, adult incontinence products, and sanitary napkins, for example. The absorbent articles typically comprise a liquid permeable topsheet, a liquid impermeable backsheet, and an absorbent core positioned at least partially intermediate the topsheet and the backsheet. Various components of absorbent articles comprise nonwoven laminates such as front and/or back ears, leg cuffs, elastic belts, and waistbands. Typically, the nonwoven laminates of these components comprise an outer nonwoven web and an inner nonwoven web with a plurality of elastic strands disposed therebetween. Elastic strands in these laminates are often visually perceptible from the garment-facing surface and can cause the absorbent article to appear diaper-like. For pull-on absorbent articles intended for children, the more of a garment (or underwear) like look that an absorbent article possesses, the more likely that a child will be willing to accept utilizing the product during the toilet training process. Likewise, for pull-on absorbent articles intended for adults experiencing incontinence, an underwear like appearance, as opposed to an overall white diaper-like appearance, may have a significant psychological influence on the adult and therefore be important in gaining the adult's acceptance in using the absorbent article. Some current absorbent articles utilize black solid colored nonwoven webs to attempt to hide the visual appearance of the elastic strands within the laminate. However, some consumers may not prefer dark, solid colored absorbent articles. For instance, consumers looking for discretion often prefer lighter and/or more neutral colored absorbent articles with a garment-like appearance. In view of the foregoing, nonwoven laminates for absorbent articles should be improved.

### SUMMARY

To solve the problems advanced above, the present disclosure provides nonwoven laminates having a disruptive coloring effect that masks and/or camouflages the visual perception of the elastic strands of the laminate in both a relaxed (pre-wear) state and in an extended (wear) state and creates a garment-like appearance.

An absorbent article comprises: a garment-facing surface; a wearer-facing surface; a topsheet; a backsheet; an absorbent core positioned at least partially intermediate the topsheet and the backsheet; and a component comprising a laminate. The laminate comprises a first nonwoven substrate, a second nonwoven substrate, and a plurality of elastic strands positioned at least partially intermediate the first nonwoven substrate and the second nonwoven substrate. The first nonwoven substrate comprises a first plurality of fibers having a first color and a second plurality of fibers having a second color, wherein the first color and the second color are different. The laminate comprises a first side and a second side opposite the first side, wherein the first nonwoven substrate forms a portion of the first side. The first side of the laminate comprises a plurality of disruptive coloring clusters.

An absorbent article comprises: a garment-facing surface; a wearer-facing surface; a topsheet; a backsheet; an absorbent core positioned at least partially intermediate the topsheet and the backsheet; and a component comprising a laminate. The laminate comprises a first nonwoven substrate, a second nonwoven substrate, and a plurality of elastic strands positioned at least partially intermediate the first nonwoven substrate and the second nonwoven substrate. The first nonwoven substrate comprises a first plurality of fibers having a first color and a second plurality of fibers having a second color, wherein the first color and the second color are different. The laminate comprises a first side and a second side opposite the first side, wherein the first nonwoven substrate forms a portion of the first side. The first side of the laminate comprises a plurality of disruptive coloring clusters. The disruptive coloring clusters have a first maximum delta E in a relaxed state and a second maximum delta E in an extended state, as measured according to the Disruptive Coloration Test Method. The first maximum delta E is greater than the second maximum delta E.

An absorbent article comprises: a garment-facing surface; a wearer-facing surface; a topsheet; a backsheet; an absorbent core positioned at least partially intermediate the topsheet and the backsheet; and a component comprising a laminate. The laminate comprises a first nonwoven substrate, a second nonwoven substrate, and a plurality of elastic strands positioned at least partially intermediate the first nonwoven substrate and the second nonwoven substrate. The first nonwoven substrate comprises a first plurality of fibers having a first color and a second plurality of fibers having a second color, wherein the first color and the second color are different. The laminate comprises a first side and a second side opposite the first side, wherein the first nonwoven substrate forms a portion of the first side. The first side of the laminate comprises a plurality of disruptive coloring clusters. The plurality of elastic strands intersect at least three of the disruptive coloring clusters in the extended state, as measured according to the Disruptive Coloration Test Method.

### BRIEF DESCRIPTION OF THE DRAWINGS

The patent or application file contains at least one photograph executed in color. Copies of this patent or patent application publication with color photograph(s) will be provided by the Office upon request and payment of the necessary fee.

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is an image of a nonwoven substrate taken by light microscopy at 200× magnification showing a first plurality of fibers having a first color and a second plurality of fibers having a second color;

FIG. 2A is a cross-sectional view of an example laminate as described herein;

FIG. 2B is a cross-sectional view of another example laminate as described herein;

FIG. 3 is a plan view of an example absorbent article in the form of a taped diaper, garment-facing surface facing the viewer, in a flat laid-out state;

FIG. 4 is a plan view of the example absorbent article of FIG. 3, wearer-facing surface facing the viewer, in a flat laid-out state;

FIG. 5 is a front perspective view of the absorbent article of FIGS. 3 and 4 in a fastened position;

FIG. 6 is a front perspective view of an absorbent article in the form of a pant;

FIG. 7 is a rear perspective view of the absorbent article of FIG. 6;

FIG. 8 is a plan view of the absorbent article of FIG. 6, laid flat, with a garment-facing surface facing the viewer;

FIG. 9 is a cross-sectional view of the absorbent article taken about line 9-9 of FIG. 8;

FIG. 10 is a cross-sectional view of the absorbent article taken about line 10-10 of FIG. 8;

FIG. 11 is a perspective view of an absorbent article in the form of a pant with a continuous outer cover material in a pre-fastened configuration;

FIG. 12 is a plan view of an absorbent article with a continuous outer cover material with the garment-facing surface facing the viewer;

FIG. 13 is a plan view of an example absorbent core or an absorbent article;

FIG. 14 is a cross-sectional view, taken about line 14-14, of the absorbent core of FIG. 13;

FIG. 15 is a cross-sectional view, taken about line 15-15, of the absorbent core of FIG. 13;

FIG. 16 is a plan view of an example absorbent article of the present disclosure that is a sanitary napkin;

FIG. 17 is a plan view of an example absorbent article in the form of a taped diaper, garment-facing surface facing the viewer, in a flat laid-out state.

FIGS. 18A-B are photographs of the laminate of Example 1, garment-facing surface facing the viewer, in a relaxed state showing a plurality of disruptive coloring clusters;

FIG. 18C is a Cluster Map of FIG. 18A resulting from the Disruptive Coloration Test Method;

FIGS. 19A-B are photographs of Example 1, garment-facing surface facing the viewer, in an extended state showing a plurality of disruptive coloring clusters; and

FIG. 19C is a Cluster Map of FIG. 19A resulting from the Disruptive Coloration Test Method.

### DETAILED DESCRIPTION

### Definitions

The following term explanations may be useful in understanding the present disclosure:

“Absorbent article” refers to devices, which absorb and contain body exudates and, more specifically, refers to devices, which are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, menstrual pads and the like.

“Body-facing” (also referred to as “wearer-facing”) and “garment-facing” refer respectively to the relative location of an element or a surface of an element or group of elements. “Body-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

“Longitudinal” refers to a direction running substantially perpendicular from a waist edge to an opposing waist edge of the article and generally parallel to the maximum linear dimension of the article. Directions within 45 degrees of the longitudinal direction are considered to be “longitudinal.”

“Lateral” refers to a direction running from a longitudinally extending side edge to an opposing longitudinally extending side edge of the article and generally at a right angle to the longitudinal direction. Directions within 45 degrees of the lateral direction are considered to be “lateral.”

“Disposed” refers to an element being located in a particular place or position.

“Joined” encompasses configurations whereby an element is directly secured to another element by affixing the element directly to the other element, and configurations whereby an element is indirectly secured to another element by affixing the element to intermediate member(s), which, in turn are affixed to the other element.

“Elastic,” “elastomer,” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force. Elastomeric materials may include elastomeric films, scrims, nonwovens, ribbons, strands and other sheet-like structures, and combinations thereof.

“Pre-strain” refers to the strain imposed on an elastic or elastomeric material prior to combining it with another element of the elastomeric laminate or the absorbent article. Pre-strain is determined by the following equation Pre-strain=((extended length of the elastic-relaxed length of the elastic)/relaxed length of the elastic)*100.

“Decitex” also known as Dtex is a measurement used in the textile industry used for measuring yarns or filaments. 1 Decitex=1 gram per 10,000 meters. In other words, if 10,000 linear meters of a yarn or filament weights 500 grams that yarn or filament would have a decitex of 500.

As used herein, the term “Color” comprises any color, e.g., white, black, red, blue, violet, orange, yellow, green, and indigo as well as any declination thereof or mixture thereof. Colors can be measured according to an internationally recognized 3D solid diagram of colors where all colors that are perceived by the human eye are converted into a numerical code. This system is based on three dimensions (x,y,z) and specifically L*, a*, b*. When a color is defined according to this system, L* represents lightness (0=black, 100=white), a* and b* independently each represent a two-color axis, a* representing the axis red/green (+a=red, −a=green), while b* represents the axis yellow/blue (+b=yellow, −b=blue).

As used herein, the terms “fiber” and “filament” refer to any type of artificial continuous strand produced through a spinning process, a meltblowing process, a melt fibrillation or film fibrillation process, or an electrospinning production process, or any other suitable process to make filaments. The term “continuous” within the context of filaments is distinguishable from staple length fibers in that staple length fibers are cut to a specific target length. In contrast, “continuous filaments” are not cut to a predetermined length. Instead, they can break at random lengths, but are usually longer than staple length fibers.

“Substrate” is used herein to describe a material which is primarily two-dimensional (i.e. in an XY plane) and whose thickness (in a Z direction) is relatively small (i.e. 1/10 or less) in comparison to its length (in an X direction) and width (in a Y direction). Non-limiting examples of substrates include a web, layer or layers of fibrous materials, nonwovens, films and foils such as polymeric films or metallic foils. These materials may be used alone or may comprise two or more layers laminated together. As such, a web is a substrate.

“Nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

As used herein, the term “meltblown” refers to fibers formed by extruding a molten thermoplastic material through a plurality of fine, usually circular, die capillaries as molten threads or filaments into a high velocity gas (e.g., air) stream which attenuates the filaments of molten thermoplastic material to reduce their diameter, which may be to a microfiber diameter. Thereafter, the meltblown fibers are carded by the high velocity gas stream and are deposited on a collecting surface to form a web of randomly dispersed meltblown fibers.

As used herein, the term “spunbond” refers to small diameter fibers which are formed by extruding a molten thermoplastic material as filaments from a plurality of fine, usually circular, capillaries of a spinneret with the diameter of the extruded filaments then being rapidly reduced as by, for example, eductive drawing or other well-known spunbonding mechanisms.

“Machine direction” (MD) is used herein to refer to the direction of material flow through a process. In addition, relative placement and movement of material can be described as flowing in the machine direction through a process from upstream in the process to downstream in the process.

“Cross direction” (CD) is used herein to refer to a direction that is generally perpendicular to the machine direction.

“Taped diaper” (also referred to as “open diaper”) refers to disposable absorbent articles having an initial front waist region and an initial back waist region that are not fastened, pre-fastened, or connected to each other as packaged, prior to being applied to the wearer. A taped diaper may be folded about the lateral centerline with the interior of one waist region in surface to surface contact with the interior of the opposing waist region without fastening or joining the waist regions together. Example taped diapers are disclosed in various suitable configurations U.S. Pat. Nos. 5,167,897; 5,360,420; 5,599,335; 5,643,588; 5,674,216; 5,702,551; 5,968,025; 6,107,537; 6,118,041; 6,153,209; 6,410,129; 6,426,444; 6,586,652; 6,627,787; 6,617,016; 6,825,393; and 6,861,571; and U.S. Patent Publication Nos. 2013/0072887 A1; 2013/0211356 A1; and 2013/0306226 A1.

“Pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be pre-formed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be pre-formed anywhere along the circumference of the article in the waist region (e.g., side fastened or seamed, front waist fastened or seamed, rear waist fastened or seamed). Example diaper pants in various configurations are disclosed in U.S. Pat. Nos. 4,940,464; 5,092,861; 5,246,433; 5,569,234; 5,897,545; 5,957,908; 6,120,487; 6,120,489; 7,569,039 and U.S. Patent Publication Nos. 2003/0233082 A1; 2005/0107764 A1, 2012/0061016 A1, 2012/0061015 A1; 2013/0255861 A1; 2013/0255862 A1; 2013/0255863 A1; 2013/0255864 A1; and 2013/0255865 A1, all of which are incorporated by reference herein.

Various non-limiting forms of the present disclosure will now be described to provide an overall understanding of the principles of the structure, function, manufacture, and use of the garment-like absorbent articles disclosed herein. One or more examples of these non-limiting forms are illustrated in the accompanying drawings. Those of ordinary skill in the art will understand that the garment-like absorbent articles described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

The present disclosure relates to nonwoven laminates having a disruptive coloring effect that can mask and/or camouflage the visual perception of the elastic strands in the laminate, and absorbent articles having components which comprise such laminates. To produce the disruptive coloring effect, the laminate structure may comprise a first nonwoven substrate and a second nonwoven substrate with a plurality of elastic strands disposed between the first nonwoven substrate and the second nonwoven substrate. At least one of the first nonwoven substrate and the second nonwoven substrate may comprise a first plurality of fibers having a first color and a second plurality fibers having a second color, wherein the first color is different than the second color. The first plurality of fibers and the second plurality of fibers of the nonwoven substrate may cooperate to form a non-uniform color distribution, that when combined in a laminate structure with another nonwoven substrate having a non-uniform color distribution or a solid color and a plurality of elastic strands, can create a disruptive coloring effect that gives the appearance of false edges and boundaries that hinders the detection or recognition of the elastic strands' true outline and shape in the nonwoven laminate and creates a garment-like appearance.

Without being limited by theory, it is believed that a masking effect and/or camouflage can be achieved through disruptive coloring of a surface, which is a set of markings, or clusters of varying colors, that creates the appearance of false edges and boundaries and hinders the detection or recognition of an object's true outline and shape as it intersects the markings. For example, disruptive coloring clusters in a nonwoven laminate can break up the regularity and linearity of elastic strands within a nonwoven laminate, thereby hindering its perception.

There are three main principles of disruptive coloration. The first principle is maximum disruptive contrast, which predicts that in effective disruptive patterns the adjacent elements, or disruptive coloring clusters, should contrast strongly. A strong color contrast can be measured by the color difference, or delta E, between color clusters present on the surface. The main function of high color contrast between disruptive coloring clusters is to break up the outline or the continuity of the surface. The second principle deals with the geometric relationship between adjacent elements of the color pattern. The elements, or disruptive coloring clusters, should be variable in size and shape to avoid any geometric regularities or repetitions in the pattern, mimicking natural, variable backgrounds, which would facilitate detection. Avoidance of symmetric and repetitive surface patterns can reduce the perception of an object having a high degree of regularity or symmetry, such as elastic strands in a nonwoven laminate. Finally, the third principle is differential blending, which arises when some coloring clusters of a surface pattern are similar to the color of an object allowing it to sometimes blend into the background, while other times allowing it to stand out from it. This process can break up the visible continuity of an object thereby reducing its detection.

The inventors have surprisingly found that when one or more nonwoven substrates comprising at least two different colored fibers are formed into a laminate structure, disruptive coloring clusters are formed which more effectively mask and/or camouflage the appearance of the elastic strands than when two single colored nonwoven webs are combined to form a laminate. It is further believed that the rugosities formed by the attachment and contraction of the elastic strands to the nonwoven substrates may further enhance the disruptive coloring effect and help to camouflage the elastic strands and create a garment-like appearance.

### Nonwoven Substrates

Nonwoven substrates are useful in many industries, such as the hygiene industry, the dusting and cleaning implement industry, and the healthcare industry, for example. In the hygiene industry, nonwoven substrates are used in the absorbent article field, such as use as components in diapers, pants, adult incontinence products, tampons, sanitary napkins, absorbent pads, bed pads, wipes, and various other products. Nonwoven substrates may be used in diapers, pants, adult incontinence products, and/or sanitary napkins, for example, as topsheets, back sheets, outer cover nonwoven materials, portions of leg cuffs, portions of elastic belts, acquisition materials, core wrap materials, portions of ears and side panels, portions of fastener tabs, portions of elastic waistbands, and/or secondary topsheets, for example. The nonwoven substrates of the present disclosure are not limited to any certain industry or application, but can have application across many industries and applications. The nonwoven substrates of the present disclosure may have particular application as part of a laminate useful for front and/or back elastic belts, front and/or back ears, elastic waistbands, and/or leg cuffs.

### Fiber Composition

The fibers of the nonwoven substrates of the present disclosure may comprise any suitable thermoplastic polymers. Example thermoplastic polymers are polymers that melt and then, upon cooling, crystallize or harden, but that may be re-melted upon further heating. Suitable thermoplastic polymers may have a melting temperature (also referred to as solidification temperature) from about 60° C. to about 300° C., from about 80° C. to about 250° C., or from about 100° C. to about 215° C., specifically reciting all 0.5° C. increments within the specified ranges and all ranges formed therein or thereby. And, the molecular weight of the thermoplastic polymer may be sufficiently high to enable entanglement between polymer molecules and yet low enough to be melt spinnable.

The thermoplastic polymers may be derived from any suitable material including renewable resources (including bio-based and recycled materials), fossil minerals and oils, and/or biodegradable materials. Some suitable examples of thermoplastic polymers include polyolefins, polyesters, polyamides, copolymers thereof, and combinations thereof. Some example polyolefins include polyethylene or copolymers thereof, including low density, high density, linear low density, or ultra-low density polyethylenes such that the polyethylene density ranges between about 0.90 grams per cubic centimeter to about 0.97 grams per cubic centimeter or between about 0.92 and about 0.95 grams per cubic centimeter, for example. The density of the polyethylene may be determined by the amount and type of branching and depends on the polymerization technology and co-monomer type. Polypropylene and/or polypropylene copolymers, including atactic polypropylene; isotactic polypropylene, syndiotactic polypropylene, and combination thereof may also be used. Polypropylene copolymers, especially ethylene may be used to lower the melting temperature and improve properties. These polypropylene polymers may be produced using metallocene and Ziegler-Natta catalyst systems. These polypropylene and polyethylene compositions may be combined together to optimize end-use properties. Polybutylene is also a useful polyolefin and may be used in some forms. Other suitable polymers include polyamides or copolymers thereof, such as Nylon 6, Nylon 11, Nylon 12, Nylon 46, Nylon 66; polyesters or copolymers thereof, such as maleic anhydride polypropylene copolymer, polyethylene terephthalate; olefin carboxylic acid copolymers such as ethylene/acrylic acid copolymer, ethylene/maleic acid copolymer, ethylene/methacrylic acid copolymer, ethylene/vinyl acetate copolymers or combinations thereof; polyacrylates, polymethacrylates, and their copolymers such as poly(methyl methacrylates).

The fibers of the nonwoven substrate may comprise monocomponent fibers, bi-component fibers, and/or bi-constituent fibers, round fibers or non-round fibers (e.g., capillary channel fibers), and may have major cross-sectional dimensions (e.g., diameter for round fibers) ranging from about 0.1 microns to about 500 microns. The fibers may also be a mixture of different fiber types, differing in such features as chemistry (e.g. polyethylene and polypropylene), components (mono- and bi-), decitex, shape (i.e. capillary and round), and the like. The fibers may have a fiber diameter of from about 0.5 to about 15 DTex, or about 0.5 to about 10 DTex, or about 0.5 to about 5 DTex, or about 0.8 to about 4 DTex, or about 0.8 to about 3 DTex, or about 0.8 to about 2 DTex, or about 0.8 to about 1.5 DTex, or about 1 to about 1.4 DTex, or about 1.1 to about 1.3 DTex, or about 1.2 DTex, specifically reciting all 0.1 DTex increments within the specified ranges and all ranges formed therein or thereby.

Example nonwoven substrates are contemplated where a first plurality of fibers and/or a second plurality of fibers comprise additives in addition to their constituent chemistry. For example, suitable additives include additives for antistatic properties, lubrication, softness, hydrophilicity, hydrophobicity, and the like, and combinations thereof. These additives may generally be present in an amount less than about 5 weight percent and more typically less than about 2 weight percent or less.

As used herein, the term “monocomponent fiber(s)” refers to a fiber formed from one extruder using one or more polymers. This is not meant to exclude fibers formed from one polymer to which small amounts of additives have been added for coloration, antistatic properties, lubrication, hydrophilicity, etc.

As used herein, the term “bi-component fiber(s)” refers to fibers which have been formed from at least two different polymers extruded from separate extruders but spun together to form one fiber. Bi-component fibers are also sometimes referred to as conjugate fibers or multicomponent fibers. The polymers are arranged in substantially constantly positioned distinct zones across the cross-section of the bi-component fibers and extend continuously along the length of the bi-component fibers. The configuration of such a bi-component fiber may be, for example, a sheath/core arrangement wherein one polymer is surrounded by another, or may be a side-by-side arrangement, a pie arrangement, or an “islands-in-the-sea” arrangement. Some specific examples of fibers which may be used in the nonwoven substrate may include polyethylene/polypropylene side-by-side bi-component fibers. Another example is a polypropylene/polyethylene bi-component fiber where the polyethylene is configured as a sheath and the polypropylene is configured as a core within the sheath. Still another example is a polypropylene/polypropylene bi-component fiber where two different propylene polymers are configured in a side-by-side configuration. Additionally, forms are contemplated where the fibers of a nonwoven substrate are crimped. Bi-component fibers may comprise two different resins, e.g. a first polypropylene resin and a second polypropylene resin. The resins may have different melt flow rates, molecular weights, or molecular weight distributions. Ratios of the two different polymers may be about 50/50, 60/40, 70/30, 80/20, or any ratio within these ratios. The ratio may be selected to control the amount of crimp, strength of the nonwoven layer, softness, bonding, or the like.

As used herein, the term “non-round fiber(s)” describes fibers having a non-round cross-section, and includes “shaped fibers” and “capillary channel fibers.” Such fibers may be solid or hollow, and they may be tri-lobal, delta-shaped, and may be fibers having capillary channels on their outer surfaces. The capillary channels may be of various cross-sectional shapes such as “U-shaped”, “H-shaped”, “C-shaped” and “V-shaped”. One practical capillary channel fiber is T-401, designated as 4DG fiber available from Fiber Innovation Technologies, Johnson City, TN. T-401 fiber is a polyethylene terephthalate (PET polyester).

### Nonwoven Substrate Formation

The nonwoven substrates of the present invention may be selected from any suitable type of nonwoven. Suitable non-limiting examples may include spunbond substrates, spunbond-meltblown-spunbond (SMS) substrates, thermally point bonded spunbond substrates, carded nonwoven substrates, through air bonded or hydroentangled nonwoven substrates, and combinations thereof.

Nonwoven substrates of the present disclosure may comprise fibers made by a spunmelt process. Briefly, the term “spunmelt” refers to a process of forming a nonwoven substrate from thin, continuous fibers produced by extruding molten polymers, for example thermoplastics, from orifices of a plate known as a spinneret or die. The continuous fibers are drawn as they cool. Spunmelt technologies may comprise both the meltblowing process and spunbonding processes. A spunbonding process may comprise supplying a molten polymer, which is then extruded under pressure through a large number of orifices in a plate known as a spinneret or die. The resulting continuous fibers are quenched and drawn by any of a number of methods. In the spunbonding process, the continuous fibers are collected as a loose nonwoven substrate upon a moving surface, such as a wire mesh conveyor belt, for example. The loose nonwoven substrate may be point bonded, where small points of a nonwoven substrate may be subjected to localized heating and/or localized pressure to consolidate the fibers of the nonwoven substrate to hold the substrate structure together. Spunbond fibers are generally continuous and may have a fiber diameter larger than 8 microns. For example, spunbond fibers may have diameters between about 8 microns and about 50 microns.

Nonwoven substrates of the present disclosure may comprise fibers made by a meltblowing process. The meltblowing process is related to the spunbonding process for forming fibers, in that a molten polymer is extruded under pressure through orifices in a spinneret or a die. In the meltblowing process, high velocity gas impinges upon and attenuates the fibers as they exit the die. The energy of this step is such that the formed fibers are greatly reduced in diameter as compared to those of the spunbonding process, and the fibers may be fractured so that micro-fibers of indeterminate length are produced. Coaxial meltblown is known in the art and is considered a form of meltblowing. Meltblown fibers may also be collected as a loose nonwoven substrate and point bonded. Meltblown fibers may have diameters of less than about 5 microns. For example, meltblown fibers may have a diameter between about 0.3 microns to about 5 microns.

The nonwoven substrate may also be made by using processes described in U.S. Pat. No. 11,236,448, which is hereby incorporated by reference.

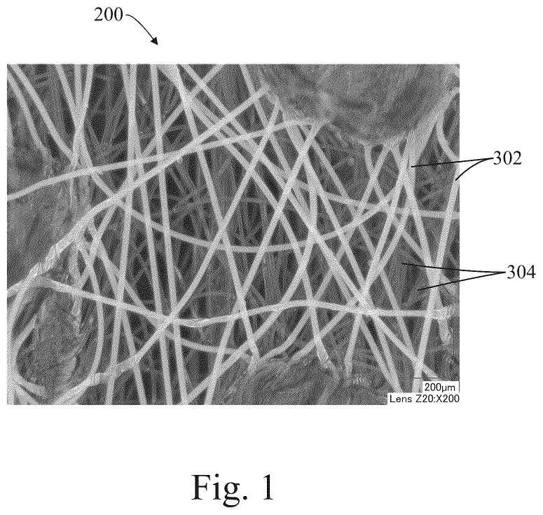

FIG. 1 presents an image of an exemplary nonwoven substrate 200 taken by light microscopy at 200× magnification. The nonwoven substrate 200 may comprise a first plurality of fibers 302 having a first color and a second plurality of fibers 304 having a second color, wherein the first color is different than the second color. In the example shown in FIG. 1, the nonwoven substrate comprises a first plurality of fibers 302 that are white in color and a second plurality of fibers 304 that are black in color. It is to be appreciated that the fibers and colors shown in FIG. 1 are for illustrative purposes and are not meant to limit the scope of the fibers and/or colors suitable for the nonwoven substrate described herein.

The fibers of the nonwoven substrate may comprise one or more colorants to achieve a color difference as provided herein. In some configurations, the first plurality of fibers and the second plurality of fibers may differ in color. The fibers of the nonwoven substrate may be differentially colored by varying the colorant composition, colorant concentration, fiber diameter, fiber density, fiber spacing, and combinations thereof. In some configurations, a first plurality of fibers may comprise a colorant while a second and/or third plurality of fibers do not comprise a colorant. In some configurations, the first plurality of fibers and the second plurality of fibers may comprise the same colorant but at different concentrations, thus creating fibers having different color intensities. For instance, the first plurality of fibers may comprise a first concentration of a colorant and the second plurality of fibers may comprise a second concentration of the same colorant, resulting in the second plurality of fibers having a greater color intensity than the first plurality of fibers. In some configurations, the colorant may be added uniformly throughout the fibers.

In some configurations, the delta E between the first color of the first plurality of fibers 302 and the second color of the second plurality of fibers 304 may be greater than 5, or greater than 7, or greater than 9, or greater than 12. In some configurations, the delta E between the first color of the first plurality of fibers 302 and the second color of the second plurality of fibers 304 may be from about 5 to about 300, or from about 5 to about 200, or from about 5 to about 100. Delta E color differences between two different colored fibers are calculated using color values acquired on a HunterLab spectrophotometer of nonwovens produced with the single-color fibers. The color measurements are made on sample stacks of ten layers of the single-color nonwoven substrates. Without being limited by theory, it is believed that having a delta E between the first color of the first plurality of fibers 302 and the second color of the second plurality of fibers 304 as described herein helps to create a nonwoven substrate having a non-uniform color distribution. If the delta E between the first color of the first plurality of fibers 302 and the second color of the second plurality of fibers 304 is less than 5, the desired non-uniform color distribution of the nonwoven substrate and/or the disruptive coloring effect of the laminate may not be achieved.

In some configurations, the nonwoven substrate 200 may optionally comprise a third plurality of fibers having a third color that is different from the first color and the second color. For example, the first plurality of fibers may be pink, the second plurality of fibers may be teal, and the third plurality of fibers may be yellow. The delta E between the third color of the third plurality of fibers and the first color of the first plurality of fibers and/or the second color of the second plurality of fibers may be greater than 5, or from about 5 to about 300.

In some configurations, one color may be white and the other colors may be non-white. In some configurations, the first color, the second color, and the third color may be non-white colors. In some configurations, the first color may be white and the second color and the third color may be non-white colors. Non-limiting examples of non-white colors can include black, blue, green, yellow, red, pink, teal, purple, orange, coral, grey, and clear. Fibers having a white color may be achieved through adding a colorant, such as titanium dioxide (TiO2) pigment, calcium carbonate pigment, and the like. In some configurations, the first color, the second color, and optionally the third color may cooperate to produce additional color(s) that are different from the first color, second color, and third color.

The fibers of the nonwoven substrate 200 may have a diameter of from about 0.3 μm to about 30 μm, or about 1 μm to about 25 μm, or about 2 μm to about 22 μm, or from about 3 μm to about 20 μm, specifically reciting all values within these ranges and any ranges created thereby. In some configurations, the first, second, and/or third plurality of fibers may have the same fiber diameter. In some configurations, the first, second, and/or third plurality of fibers may have different fiber diameters. Without being limited by theory, it is believed that by varying the fiber diameter of the first, second, and/or third plurality of fibers, nonwoven substrates can be created that mimic various textile garment appearances.

In some configurations, the basis weight of the nonwoven substrate 200 may be from about 10 grams per square meter (gsm) to about 45 gsm, or from about 15 gsm to about 30 gsm, or from about 18 gsm to about 25 gsm. Without being limited by theory, it is believed that by having a basis weight of the nonwoven substrate of from about 10 gsm to about 45 gsm, the desired non-uniform color distribution can be created that when combined in a laminate structure can create a disruptive coloring effect that effectively masks the appearance of the elastic strands.

In some configurations, the nonwoven substrate 200 may comprise from about 15% to about 85%, or from about 20% to about 70%, or from about 25% to about 50%, of the first plurality of fibers, the second plurality of fibers and/or the third plurality of fibers, specifically reciting all values within these ranges and any ranges created thereby.

In some configurations, the first plurality of fibers, the second plurality of fibers, and/or the third plurality of fibers may have a basis weight of between about 2 gsm and about 40 gsm, or between about 4 gsm and about 25 gsm, between about 5 gsm and about 15 gsm, specifically reciting all values within these ranges and any ranges created thereby.

In some configurations, the nonwoven substrate may comprise a ratio of the first plurality of fibers to the second plurality of fibers of from about 4:1 to about 1:4, or from about 2:1 to about 1:2. In some configurations, the nonwoven substrate may comprise a ratio of the first plurality of fibers to the second plurality of fibers of about 1:1. In some configurations, the nonwoven substrate may comprise a ratio of the first plurality of fibers to the second plurality of fibers to the third plurality of fibers of about 1:1:1.

In some configurations, the first plurality of fibers, the second plurality of fibers, and the third plurality of fibers may be continuous fibers that intersect or overlap one another.

A number of other fibers other than the first, second, and third plurality of fibers may be part of the nonwoven substrate 200. For instance, the nonwoven substrate 200 may comprise a fourth plurality of fibers, a fifth plurality of fibers, and/or a six plurality of fibers. For brevity, the fourth, fifth and sixth plurality of fibers will not be discussed in detail, but it is to be appreciated that such additional fibers may have the features discussed herein and each of the additional fibers may differ in color from any of the first, second, and/or third plurality of fibers.

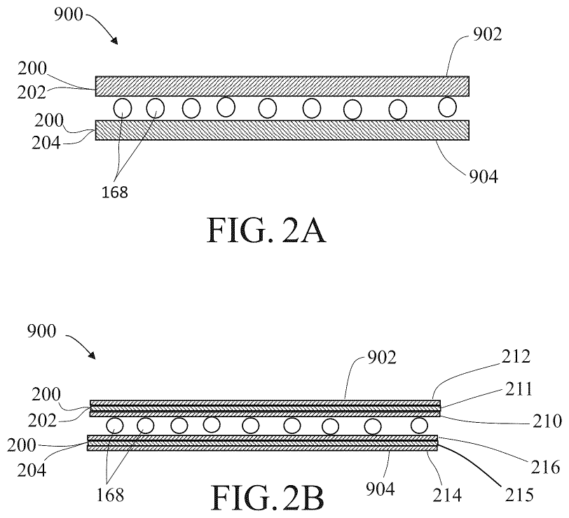

FIGS. 2A and 2B are cross-sectional views of a laminate 900 of the present disclosure. The laminate 900 may comprise a first nonwoven substrate 202, a second nonwoven substrate 204, and a plurality of elastic strands 168 positioned at least partially intermediate the first nonwoven substrate 202 and the second nonwoven substrate 204. The laminate 900 may comprise a first side 902 and a second side 904 opposite the first side 902. The first nonwoven substrate 202 may form a portion of the first side 902 and the second nonwoven substrate 204 may form a portion of the second side 904. It is to be understood that the description above regarding the nonwoven substrate 200 may also apply to the first nonwoven substrate 202 and the second substrate 204. The first nonwoven substrate, second nonwoven substrate, and the plurality of elastic strands may be joined using an adhesive at a basis weight of from about 10 mg/m to about 130 mg/m, or from about 15 mg/m to about 120 mg/m, specifically reciting all values within these ranges and any ranges created thereby.

In some configurations, as shown in FIG. 2A, the first and/or second nonwoven substrates 202, 204 may be a single layer material comprising a mixture of fibers. For instance, the first and/or second nonwoven substrate 202, 204 may comprise a first plurality of fibers, a second plurality of fibers, and optionally a third plurality of fibers which are bonded together, for example by thermal point bonds, to form a unitary web. In some configurations, as shown in FIG. 2B, the first and/or second nonwoven substrates 202, 204 may comprise two or more sublayers, wherein each sublayer comprises a plurality of fibers. For instance, the first nonwoven substrate 202 may comprise a first sublayer 210 comprising a first plurality of fibers, a second sublayer 211 comprising a second plurality of fibers, and optionally a third sublayer 212 comprising a third plurality of fibers, wherein the first, second, and third sublayers 210, 211, 212, are bonded together to form a unitary web. The second nonwoven substrate 204 may comprise a first sublayer 214 comprising a first plurality of fibers, a second sublayer 215 comprising a second plurality of fibers, and optionally a third sublayer 216 comprising a third plurality of fibers, wherein the first, second, and third sublayers 214, 215, 216, are bonded together to form a unitary web. The fibers of the second nonwoven substrate 204 may be the same or may be different from the fibers of the first nonwoven substrate 202.

In some configurations, the color of one sublayer may be different from the color of at least one of the other sublayer(s) to provide a non-uniform color distribution in the nonwoven substrate. In some configurations, a portion of the color of the second and/or third sublayer may be visible under normal lighting conditions through a portion of the first sublayer to provide a non-uniform color distribution. In some configurations, the basis weight of at least one of the sublayers may be less than another sublayer to allow cooperation between the respective colors of the sublayers.

The first nonwoven substrate 202 may comprise a first plurality of fibers having a first color and a second plurality of fibers having a second color, wherein the first color and the second color are different. In some configurations, the first nonwoven substrate 202 may optionally comprise a third plurality of fibers having a third color. In some configurations, the first color may be white and the second color and third color may be non-white colors. In some configurations, the first color, the second color, and the third color (if present) may be non-white colors. At least one of the first plurality of fibers, the second plurality of fibers, and the third plurality of fibers (if present) may comprise a colorant as described herein. In some configuration, the first plurality of fibers, the second plurality of fibers, and the third plurality of fibers (if present) may comprise a colorant.

The second nonwoven substrate 204 may comprise a plurality of fibers. In some configurations, the second nonwoven substrate 204 may be the same as the first nonwoven substrate 202. In some configurations, the second nonwoven substrate may be different than the first nonwoven substrate. In some configurations, the second nonwoven substrate 204 may comprise at least one of the first plurality of fibers, the second plurality of fibers, and the third plurality of fibers. In some configurations, the second nonwoven substrate may comprise fibers of substantially the same color, which may be the same as at least one of the colors of the first nonwoven substrate. In some configurations, the second nonwoven substrate may comprise fibers of substantially the same color, which may be different than the colors of the first nonwoven substrate.

In some configurations, the second nonwoven substrate 204 may comprise fibers that differ in color from the fibers of the first nonwoven substrate 202. For example, the first nonwoven substrate may comprise a first plurality of fibers having a first color, a second plurality of fibers having a second color, a third plurality of fibers having a third color, and/or a fourth plurality of fibers having a fourth color, and the second nonwoven substrate may comprise a fifth plurality of fibers having a fifth color, a sixth plurality of fibers having a sixth color, a seventh plurality of fibers having a seventh color, and/or an eighth plurality of fibers having an eighth color.

In some configurations, the first and/or second nonwoven substrate 202, 204 may comprise spunbond and/or meltblown fibers. Suitable configurations may include, for example, SS, SSS, SSSS, SMS, SMMS, and SSMMS. It is to be appreciated that at least two of the sublayers of fibers within the nonwoven substrate differ in color to provide the desired non-uniform color distribution.

In some configurations, the second nonwoven substrate may have a color that is substantially similar to the color of the plurality of elastic strands.

In some configurations, the first side 902 of the laminate 900 may form a portion of a garment-facing surface and the second side 904 of the laminate 900 may form a portion of a wearer-facing surface.

In some configurations, the first side of the laminate may comprise a plurality of disruptive coloring clusters. In some configurations, the disruptive coloring clusters may have an irregular shape and/or are positioned in a non-periodic pattern. Without being limited by theory, it is believed that by having disruptive coloring clusters having an irregular shape and/or non-periodic pattern may help to mask and/or camouflage the elastics of the laminate by mimicking natural, variable backgrounds thereby reducing the perception of an object having a high degree of regularity or symmetry, such as elastic strands in a nonwoven laminate.

In some configurations, the first side of the laminate may comprise at least one pair of disruptive coloring clusters having a delta E of greater than about 2, or greater than about 3, or greater than about 5, or greater than about 10, or greater than 15, as measured according to the Disruptive Coloration Test Method in a relaxed state and/or in an extended state. In some configurations, the first side of the laminate may comprise more than one pair, or more than two pairs, or more than three pairs, or more than four pairs, of disruptive coloring clusters having a delta E of from about 2 to about 100, or from about 3 to about 50, or from about 4 to about 25, or from about 10 to about 20, as measured according to the Disruptive Coloration Test Method in a relaxed state and/or in an extended state.

In some configurations, the first side of the laminate may comprise a plurality of disruptive coloring clusters, wherein the disruptive coloring clusters have a first maximum delta E in a relaxed state and a second maximum delta E in an extended state, as measured according to the Disruptive Coloration Test Method. The first maximum delta E may be greater than the second maximum delta E. Without being limited by theory, it is believed that by having a delta E between the disruptive coloring clusters as described herein (in both a relaxed and extended state), the elastics may be less visually perceptible and the laminate may have a more garment-like appearance both before and during wear of the absorbent article.

In some configurations, the plurality of elastic strands intersect at least 3, or at least 5, or at least 8, or at least 10, or at least 15, disruptive coloring clusters, as measured in an extended state according to the Disruptive Coloration Test Method. In some configurations, the plurality of elastic strands intersect from 3 to 30, or 5 to 25, or 10 to 20, disruptive coloring clusters, as measured in an extended state according to the Disruptive Coloration Test Method. In some configurations, at least a pair of the disruptive coloring clusters that are intersected by an elastic strand have a delta E of greater than 2, or greater than 3, or greater than 5, as measured according to the Disruptive Coloration Test Method. Each elastic strand of the laminate may encounter one or more disruptive coloring clusters as the strand extends laterally across the laminate. Without being limited by theory, it is believed that the transition between the colors of the disruptive coloring clusters that each elastic strand encounters helps to break up the visual line of the elastic strand, thus providing the masking benefit and helping to create the garment-like appearance. It was surprisingly found that effective elastic strand masking and/or camouflaging can be achieved when at least a pair of the disruptive coloring clusters that are intersected by an elastic strand have a delta E of greater than 2.

It is to be appreciated that the discussions and descriptions above regarding the disruptive coloring clusters and delta E between the disruptive coloring clusters of the first side 902 of the laminate 900 may also apply to the second side 904 of the laminate 900.

In some configurations, the laminate 900 may be sided. In some configurations, the first side 902 of the laminate 900 may comprise a first plurality of disruptive coloring clusters and the second side 904 of the laminate 900 may comprise a second plurality of disruptive coloring clusters. In some configurations, the first and second plurality of disruptive coloring clusters are substantially similar. In some configurations, the second plurality of disruptive coloring clusters are visually different in size, shape, distribution, and/or color than the first plurality of disruptive coloring clusters.

In some configurations, the first and second plurality of fibers may be substantially oriented in the machine direction within the nonwoven substrate. As used herein, substantially oriented in the MD direction means that the nonwoven substrate has a MD/CD strength ratio of greater than about 2, or greater than about 3, or greater than about 4. Without being limited by theory, it is believed that when the first and second plurality of fibers are substantially oriented in the machine direction, a striated textile-like appearance can be created in the nonwoven substrate which, when combined in a laminate, can help to break up the shape and disrupt the outline of the elastic strands in the laminate, thus hindering detection of the elastic strands.

### Colorants

The nonwoven substrates of the present disclosure may comprise a colorant which may be used in absorbent articles that are worn by consumers. Such nonwoven substrates may be used to manufacture portions of an absorbent article, such as components comprising a laminate, that are consumer pleasing and premium looking. The term “colorant”, as used herein, refers to inks, dyes, pigments, or the like, and combinations thereof, used to create color in a substrate.

Generally, colorants, such as pigments, are added to the thermoplastic material in the form of a masterbatch or concentrate at the time of formation of the fibers. For nonwoven substrates that are formed via a spunmelt process, e.g. spunbond, meltblown, etc., polymeric material is extruded through a plurality of holes in a die. Therefore, the colorant must pass through the holes in the die along with the polymeric material. The colorant may thus be locked in the polymer matrix of the filament. The use of colorants in nonwoven substrates may provide certain advantages, such as preventing colorant from rubbing off on a wearer.

A colorant masterbatch may be added to the polymer formulation of spunbond and/or meltblown fibers at about 0.25 percent by weight to about 8 percent by weight, about 0.5 percent by weight to about 6 percent by weight, about 0.75 percent by weight to about 5 percent by weight, about 1 percent by weight to about 4.5 percent by weight, about 1.25 percent by weight to about 4 percent by weight, or about 1.5 percent by weight to about 3.5 percent by weight, specifically reciting all values within these ranges and any ranges created thereby.

Some colorants may comprise particles having sizes less than about 2 μm or less than about 1 μm. In some configurations, colorants may comprise particles having sizes ranging from about 8 nm to about 100 nm.

A pigment is a material, which can be organic or inorganic and may include activatable, structural and or special effects pigments. A pigment changes the color of reflected or transmitted light as the result of wavelength-selective absorption. This physical process differs from fluorescence, phosphorescence, and other forms of luminescence, in which a material emits light. A pigment is a generally insoluble powder, which differs from a dye, which either is itself a liquid or is soluble in a solvent (resulting in a solution). The pigment will typically be mixed with the thermoplastic material, of which the fibers are made. Often, the pigment is added to the thermoplastic material in the form of a masterbatch or concentrate at the time of formation of the fibers. Colored masterbatches useful for the present invention include polypropylene based custom color masterbatches e.g., supplied by Ampacet; Lufilen and Luprofil supplied by BASF; and Remafin for polyolefin fibers, Renol-AT for polyester fibers, Renol-AN for polyamide fibers and CESA for renewable polymers supplied by Clariant. Hence, the pigment will be suspended in the molten thermoplastic material prior to the thermoplastic material being forced through the spinnerets to form and lay down the fibers which form the nonwoven substrate.

To increase the whiteness and/or opacity of the fibers, TiO2 may be used. Different crystal forms are available, however most preferred are rutile or anatase TiO2. Other white pigments include zinc oxide, zinc sulfide, lead carbonate or calcium carbonate. To create a black color, carbon black or any other suitable colorant may be used. Various colored inorganic pigments may be used depending upon the desired color and may include metal oxides, hydroxides and sulfides or any other suitable material. Non-limiting examples of inorganic pigments include cadmium orange, iron oxide, ultramarine, chrome oxide green. One or more pigments may be combined to create the desired color. Non-limiting examples of organic colorants include anthraquinone pigments, azo pigments, benzimidazolone pigments, BONA Lakes, Dioxazine, Naphthol, Perylene, Perinone, Phthalocyanine, Pyranthrone, Quinacridones. Effects pigments including metal, pearlescent and fluorescent may also be used. Various colorants are described in Plastics Additives Handbook, 5th Edition.

### General Description of an Absorbent Article

As described herein, the laminate 900 of the present disclosure may be used as one or more components of an absorbent article. An example absorbent article is set forth below.

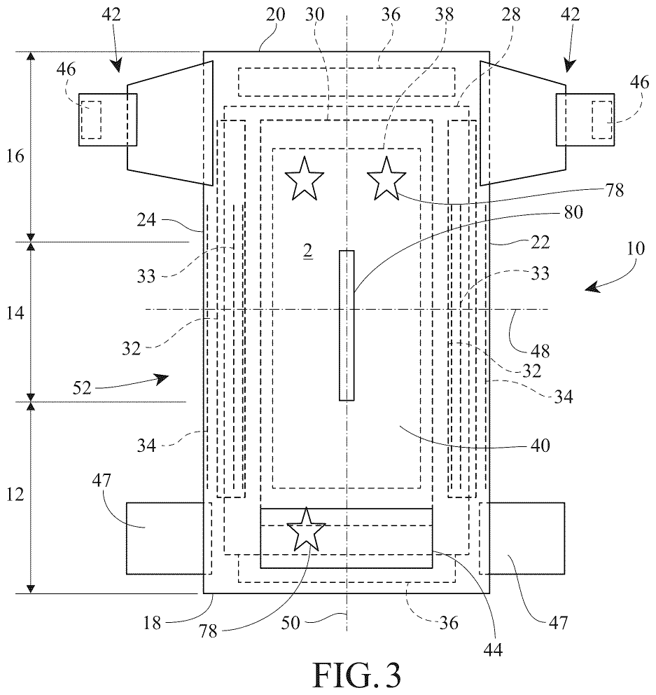

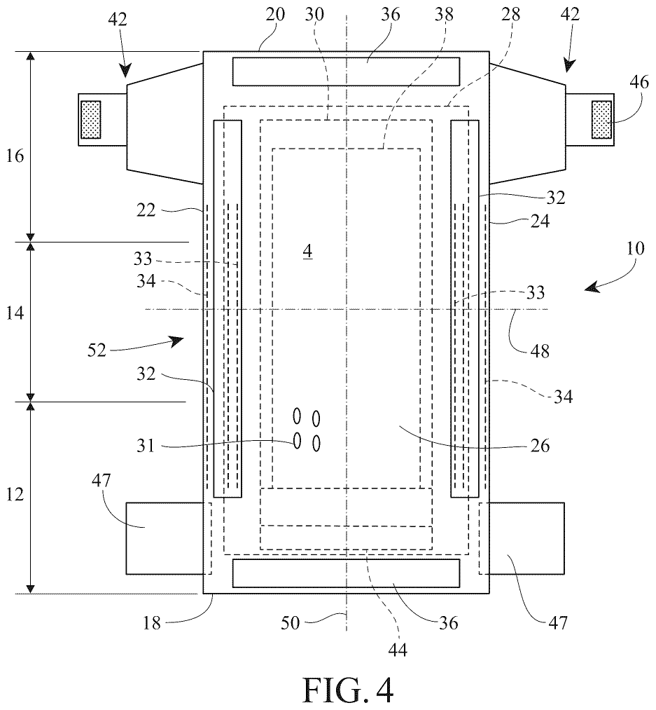

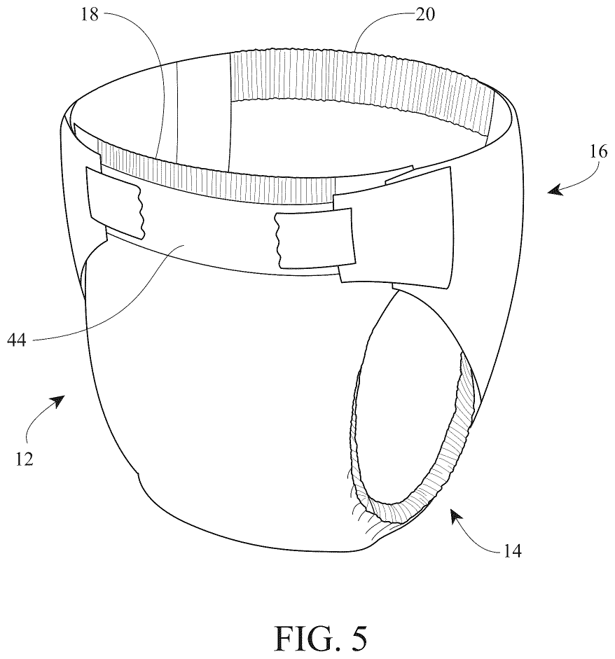

An example absorbent article 10 according to the present disclosure, shown in the form of a taped diaper, is represented in FIGS. 3-5. FIG. 3 is a plan view of the example absorbent article 10, garment-facing surface 2 facing the viewer in a flat, laid-out state (i.e., no elastic contraction). FIG. 4 is a plan view of the example absorbent article 10 of FIG. 3, wearer-facing surface 4 facing the viewer in a flat, laid-out state. FIG. 5 is a front perspective view of the absorbent article 10 of FIGS. 3 and 4 in a fastened configuration. The absorbent article 10 of FIGS. 3-5 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50.

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137.

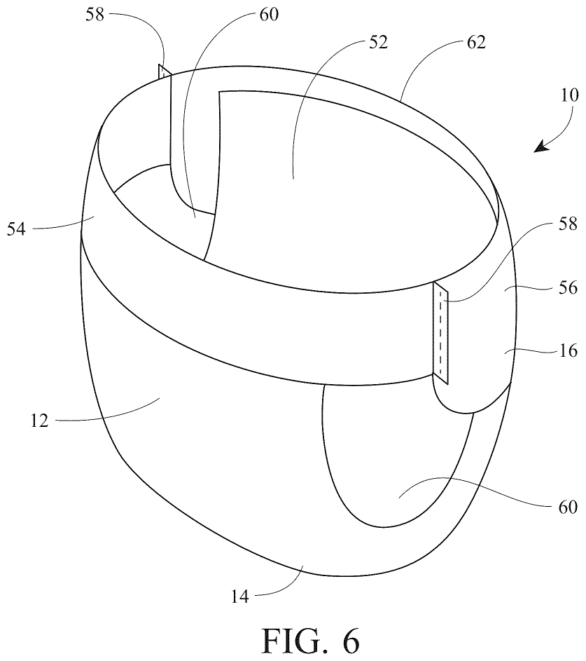

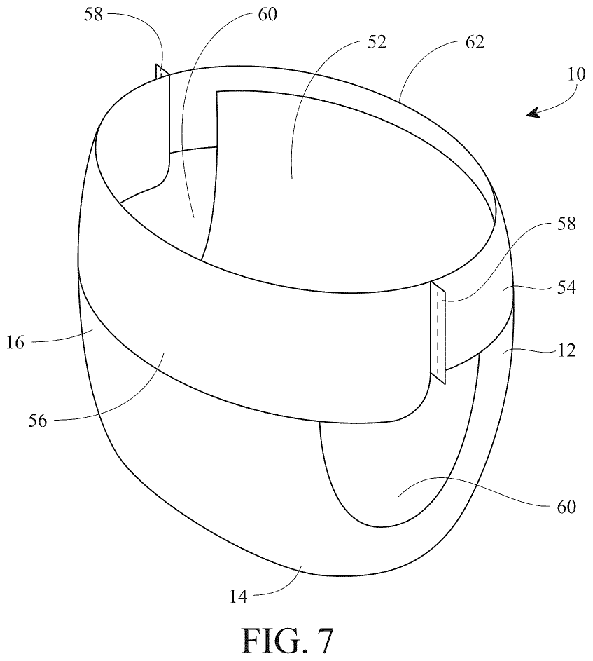

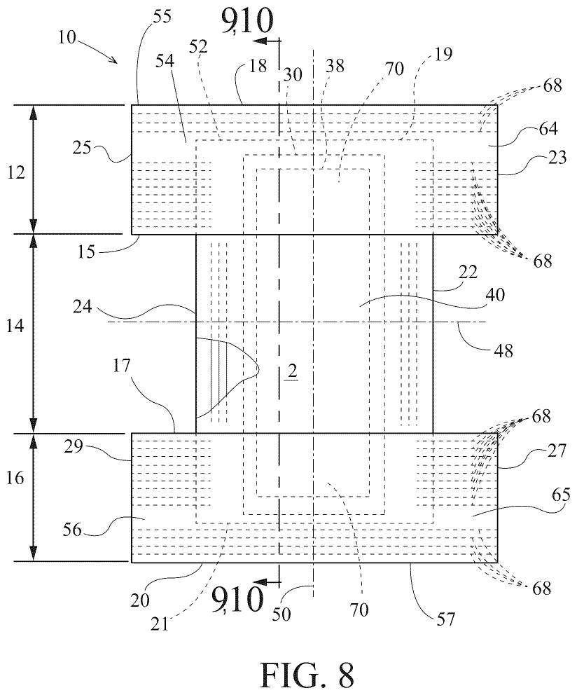

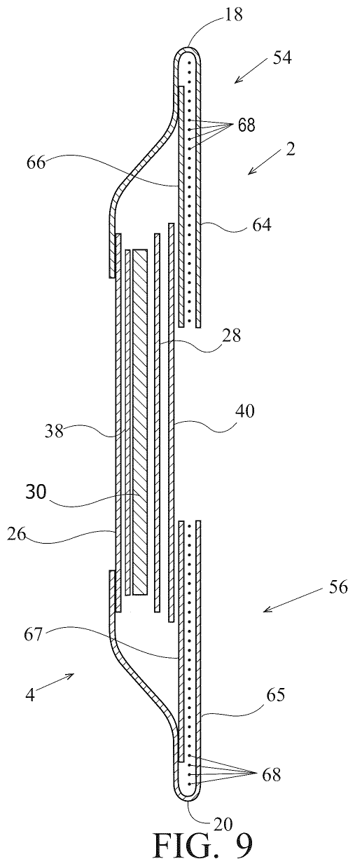

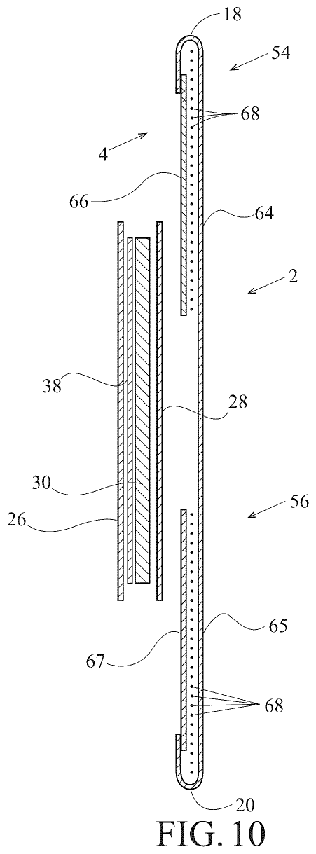

Referring to FIGS. 6-10, an example absorbent article 10 in the form of a pant is illustrated. FIG. 6 is a front perspective view of the absorbent article 10. FIG. 7 is a rear perspective view of the absorbent article 10. FIG. 8 is a plan view of the absorbent article 10, laid flat, with the garment-facing surface facing the viewer. Elements of FIG. 6-10 having the same reference number as described above with respect to FIGS. 3-5 may be the same element (e.g., absorbent core 30). FIG. 9 is an example cross-sectional view of the absorbent article taken about line 9-9 of FIG. 8. FIG. 10 is an example cross-sectional view of the absorbent article taken about line 10-10 of FIG. 8. FIGS. 9 and 10 illustrate example forms of front and back elastic belts 54, 56 (also referred to herein as front and back belts). The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 3-5. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

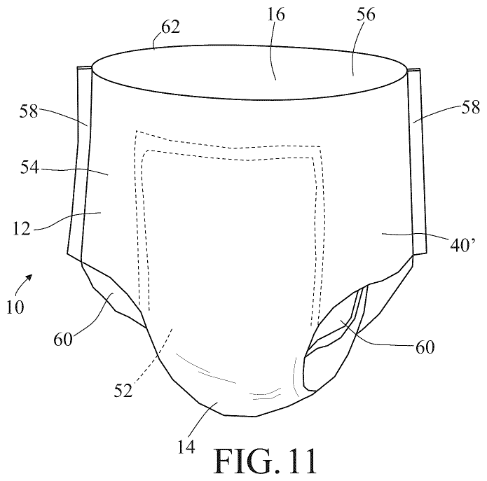

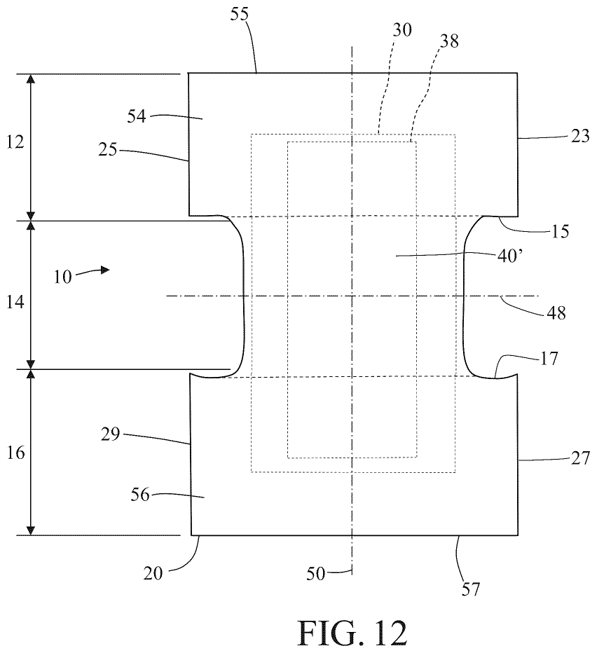

In some configurations, such as shown in FIGS. 11-12, the absorbent article 10 may comprise a continuous outer cover material 40′. FIG. 11 is a front perspective view of the absorbent article 10 in the form of a pant with a continuous outer cover material 40′. FIG. 12 is a plan view of the absorbent article 10 with a continuous outer cover material 40′, laid flat, with the garment-facing surface facing the viewer. Elements of FIG. 11-12 having the same reference number as described herein with respect to FIGS. 3-5 or 7-10 may be the same element (e.g., absorbent core 30). In some configurations, the outer belt layer may be configured to define a continuous outer cover 40′ that extends contiguously from the front belt end edge 55 to the back belt end edge 57.

### Belts

Referring to FIGS. 9 and 10, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. No. 9,072,632.

Front and back belt end edges 55 and 57 may extend longitudinally beyond the front and back chassis end edges 19 and 21 (as shown in FIG. 8) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 9 and a gap between the front and back inner belt layers 66 and 67 is shown in FIG. 10. In some configurations shown in FIGS. 9 and 10, portions of the outer belt layers 64, 65 may be folded over onto the inner belt layers, respectively. In addition, as shown in FIG. 9, portions of the outer belt layers 64, 65 may also be folded over onto the chassis 52.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 6 and 7).

The front and back belts 54 and 56 may comprise graphics (see e.g., 78 of FIG. 3). The graphics may extend substantially around the entire circumference of the absorbent article 10 and may be disposed across side seams 58 and/or across proximal front and back belt edges 15 and 17; or, alternatively, adjacent to the seams 58 and/or proximal front and back belt edges 15 and 17 in the manner described in U.S. Pat. No. 9,498,389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 4, element 31), may have any suitable three-dimensional features, and/or may have a plurality of embossments (e.g., a bond pattern). The topsheet may comprise a variable basis weight nonwoven material, such as those described in U.S. Pat. Appl. No. 2017/0191198. The topsheet may be apertured by overbonding a material and then rupturing the overbonds through ring rolling, such as disclosed in U.S. Pat. No. 5,628,097, to Benson et al., issued on May 13, 1997, and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials, such as films, which permit vapors to escape from the absorbent article, while still preventing, or at least inhibiting, bodily exudates from passing through the backsheet. The backsheet 28 may be coterminous with the outer cover material 40.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material or a variable basis weight nonwoven material.

### Absorbent Core

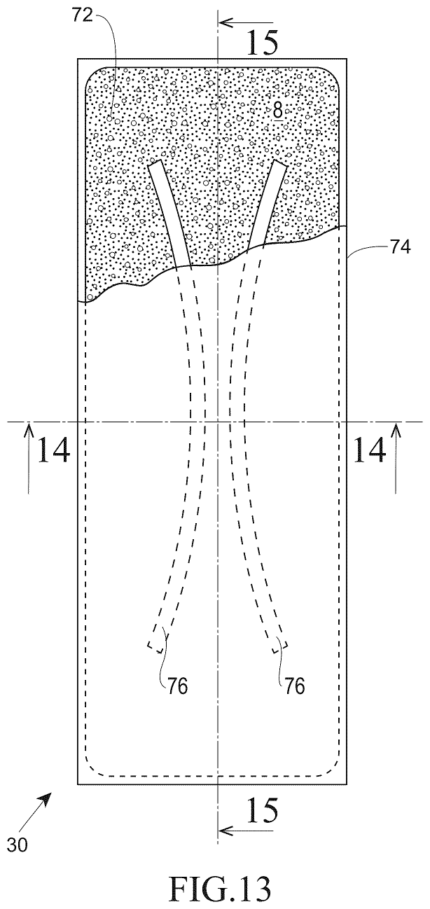

As used herein, the term “absorbent core” 30 refers to a component of the absorbent article 10 disposed in the absorbent article for absorbing and containing liquid such as urine received by the absorbent article. The absorbent core thus typically has a high absorbent capacity. An example absorbent core 30 is schematically shown in FIGS. 13-15. The absorbent core comprises an absorbent material 72, that is typically enclosed within or sandwiched between a core bag 74.

The core wrap may be a single material that is folded and attached to itself, or it may comprise a separate top layer and bottom layer that may be bonded, adhesively joined, or otherwise joined together. The top and bottom layers of the core wrap may be the same or different. The absorbent material typically comprises superabsorbent particles which are optionally mixed with cellulose fibers. As used herein, “absorbent core” does not include any acquisition-distribution systems, topsheet, or backsheet of the absorbent article.

The example absorbent core 30 shown in isolation in FIGS. 13-15 is in the dry state (before use). The absorbent core may typically have a generally rectangular shape as defined by its longitudinal edges and transversal front edge and back edge or may have other shapes.

Absorbent material 72 may be deposited as an absorbent layer having a generally rectangular outline, as represented in FIG. 13. A wide variety of absorbent cores may also be used. The absorbent material 72 layer may also have a non-rectangular perimeter (“shaped” core), in particular, the absorbent material 72 may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “hourglass” for the area of the absorbent material.

The absorbent material 72 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores, where the absorbent material consists, or consists essentially, of SAP. The absorbent material may also be a high internal phase emulsion foam.

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from about 20 g/g to about 40 g/g. “Superabsorbent polymer particles”, as used herein, refers to a superabsorbent polymer material which is in particulate form so as to be flowable in the dry state.

Various absorbent core designs comprising high amounts of SAP have been proposed in the past, see for example in U.S. Pat. No. 5,599,335 (Goldman), EP1,447,066 (Busam), WO95/11652 (Tanzer), U.S. Pat. Appl. Pub. No. 2008/0312622A1 (Hundorf), WO2012/052172 (Van Malderen). In particular, the SAP printing technology as disclosed in U.S. Pat. No. 7,838,722 (Blessing), U.S. Pat. Nos. 9,072,634 and 8,206,533 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate, such as a carded nonwoven layer, having a porous structure into which SAP particles have been deposited, may also be used in present disclosure.

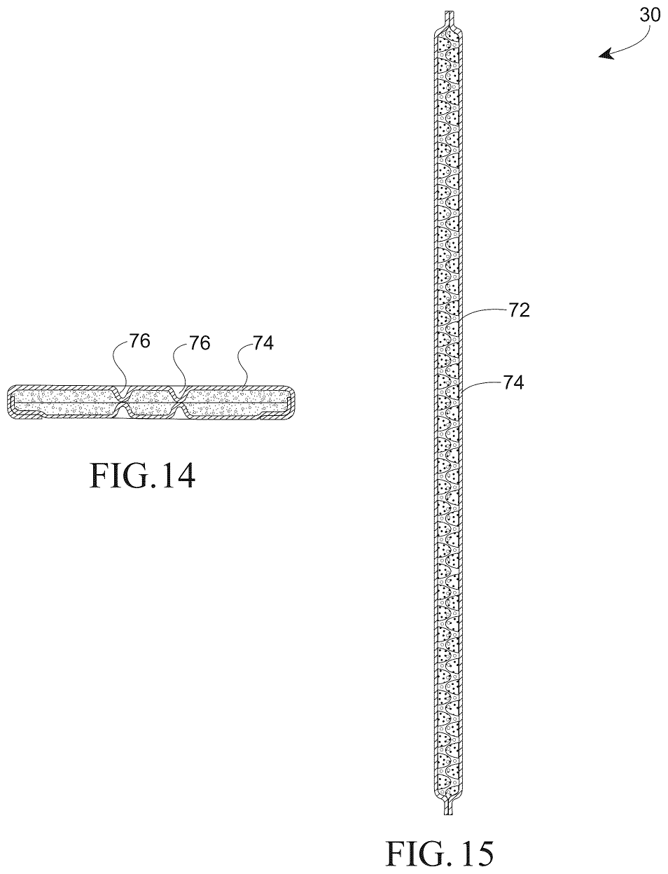

The absorbent material may be deposited as a continuous layer within the core wrap. The absorbent material may also be present discontinuously, for example, as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern, wherein the resulting layer is substantially continuously distributed across the absorbent particulate polymer material area, as illustrated in FIGS. 14-15. As for example taught in U.S. Pat. Appl. Pub. No. 2008/0312622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material within the absorbent material layer. The top side of the core wrap may be advantageously bonded to the bottom side of the core by adhesive, mechanical or ultra-sonic bonding through these material-free areas. Example disclosures of such channels in an airfelt-free core can be found in U.S. Pat. No. 9,789,011. One or more channels may also be formed in absorbent cores comprising a mix of cellulose fibers and SAP particles. These channels may embody any suitable shapes and any suitable number of channels may be provided. In other instances, the absorbent core may be embossed to create the impression of channels, with absorbent material remaining within the channels. The absorbent core in FIGS. 13-15 is merely an example absorbent core. Many other absorbent cores with or without channels are also within the scope of the present disclosure.

### Barrier Leg Cuffs/Leg Elastics

Referring to FIGS. 3 and 5, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14.

### Elastic Waistband

Referring to FIGS. 3 and 4, the absorbent article 10 may comprise one or more elastic waistbands 36. The elastic waistbands 36 may be positioned on the garment-facing surface 2 or the wearer-facing surface 4. Alternatively, the elastic waistbands 35 may be positioned intermediate the topsheet and the backsheet. As an example, a first elastic waistband 36 may be present in the front waist region 12 near the front belt end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article. The elastic waistband may comprise an elastic film joined to the topsheet and a nonwoven material covering the elastic film. In other instances, the elastic waistband may comprise an elastic film sandwiched between two nonwoven materials. The elastic film may be ultrasonically bonded, or otherwise bonded or attached, to the one or more nonwoven materials. The elastic film and/or the nonwoven materials may be preactivated (i.e., activated prior to being joined together) or the formed elastic film/nonwoven laminate may be activated post laminate formation.

### Acquisition Materials

Referring to FIGS. 3, 4, 9, and 10, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, formed films, apertured formed films, cellulosic materials, cross-linked cellulosic materials, air laid cellulosic nonwoven materials, spunlace materials, or combinations thereof, for example. The acquisition material may be rectangular or may be shaped, such as hourglass shaped. Typically, an acquisition material 38 may have a width and length that are smaller than the width and length of the topsheet 26. The acquisition material may be a secondary topsheet in the feminine pad context. The acquisition materials may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 3 and 5, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Wetness Indicator/Graphics

Referring to FIG. 3, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78.

### Front and Back Ears

Referring to FIGS. 3 and 5, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. Any ears with fasteners may be configured to engage an opposing ear or the landing zone or landing zone area 44. One set of ears may comprise primary fasteners and the other set of ears may comprise secondary fasteners. The ears, or portions thereof, may be elastic or may have elastic panels. In an example, an elastic film or elastic strands may be positioned intermediate a first nonwoven material and a second nonwoven material. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2. The back ears may comprise an elastic film sandwiched between two nonwoven materials forming a laminate. The elastic film and/or the nonwoven materials may be pre-activated (i.e., activated prior to being joined together) and the laminate may be joined by ultrasonic bonds. Such laminates are disclosed in U.S. Pat. No. 10,485,713. In other instances, the elastic film may not be preactivated and instead the laminate may be activated after the laminate is formed. Such laminates may also be ultrasonically bonded.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 74 and the liquid impermeable backsheet 28.

### Packages

The absorbent articles of the present disclosure may be placed into packages. The packages may comprise polymeric films, paper, and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages. The packages may comprise polymeric films comprising recycled material, such as about 20% to about 100%, about 30% to about 90%, about 30% to about 80%, about 40% to about 60%, or about 50% recycled material. The recycled material may comprise post-industrial recycled material (PIR) and/or post-consumer recycled material (PCR). In some instances, the polymeric films used for the packages may comprise two outer layers and one or more inner layers. The one or more inner layers may comprise the recycled material or may comprise more recycled material than the outer layers. The recycled material may comprise recycled polyethylene. The recycled material may comprise recycled polyethylene PIR from trim from the packaging operation.

The package material may comprise paper, paper based material, paper with one or more barrier layers, or a paper/film laminate. The package material may be in the range of about 50 gsm to about 100 gsm or about 70 gsm to about 90 gsm and the one or more barrier layers may be in the range of about 3 gsm to about 15 gsm. The paper based package material with or without one or more barrier layers may exhibit a machine direction tensile strength of at least 5.0 kN/m, a machine direction stretch of at least 3 percent, a cross-machine direction tensile strength of at least 3 kN/m, and a cross-direction stretch at break of at least 4 percent, each as determined via ISO 1924-3.

The paper based package material or paper based package material comprising a barrier layer or film may be recyclable or recyclable in normal paper recycling operations. The recyclability extent of the paper based package may be determined via recyclable percentage. The paper based package of the present disclosure may exhibit recyclable percentages of 70 percent or greater, 80 percent or greater, or 90 percent or greater. The paper based package of the present disclosure may have a recyclable percentage of between 70 percent to about 99.9 percent, between about 80 percent to about 99.9 percent, or between about 90 percent to about 99.9 percent. In one example, the package material of the present disclosure may exhibit a recyclable percentage of from about 95 percent to about 99.9 percent, from about 97 percent to about 99.9 percent, or from about 98 percent to about 99.9 percent. The recyclable percentage of the paper based package may be determined via test PTS-RH:021/97 (Draft October 2019) under category II, as performed by Papiertechnische Stiftung located at Pirnaer Strasse 37, 01809 Heidenau, Germany. In another instance, the paper based packages of the present disclosure may exhibit an overall “pass” test outcome as determined by PTS-RH:021/97 (Draft October 2019) under category II method. Any of the paper based packages may have opening features, such as lines of perforation, and may also have handles.

### Arrays

“Array” means a display of packages comprising disposable absorbent articles of different article constructions (e.g., different elastomeric materials [compositionally and/or structurally] in the side panels, back ears, side flaps and/or belts flaps, different graphic elements, different product structures, fasteners, waistbands, or lack thereof). The packages may have the same brand and/or sub-brand and/or the same trademark registration and/or having been manufactured by or for a common manufacturer and the packages may be available at a common point of sale (e.g. oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” distributed by a common on-line source.

### Sanitary Napkin

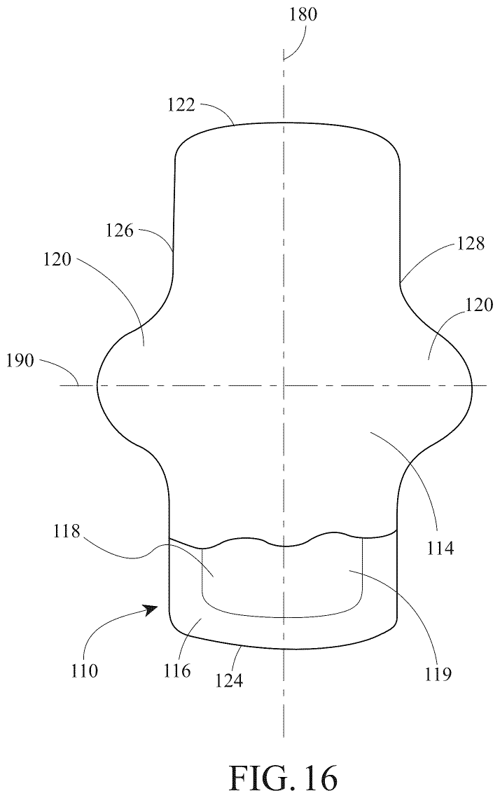

Referring to FIG. 16, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 128 to a midpoint of the second side edge 128. The sanitary napkin 110 may also be provided with additional features commonly found in sanitary napkins as is known in the art.

### Fasteners

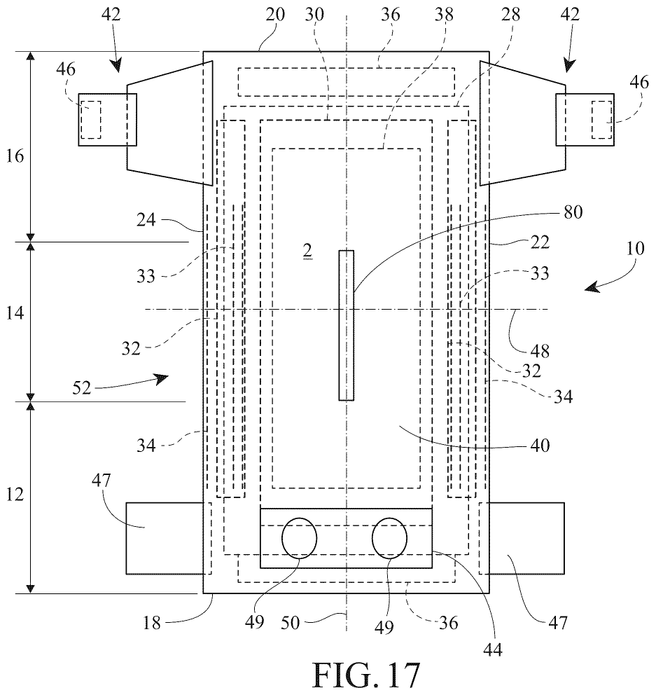

Referring to FIG. 17, the back ears 42 may comprise fasteners 46. The fasteners 46 may be configured to cooperate with the landing zone area or landing zone material 44 on the garment-facing surface of the front waist region 12 of the absorbent article 10. Additionally, the absorbent article may comprise one or more secondary fasteners 49. The secondary fasteners 49 may be disposed on the outer cover material 40 or a component of the absorbent article, such as the landing zone 44 as illustrated in FIG. 17. The secondary fasteners may be a separate material joined to the absorbent article. The secondary fasteners may be integrally formed from a material of the absorbent article. For example, the secondary fasteners may comprise one or more hooks or protrusions formed from the material of the outer cover material 40, the landing zone material 44, or a film. The hooks or protrusions may be formed using the process described in U.S. Pat. No. 8,784,722 to Rocha et al. The secondary fasteners may be configured to cooperate with a portion of the back ears. For example, a secondary fastener comprising hooks or protrusions are configured to engage the nonwoven material of the back ear. It is to be appreciated that the secondary fasteners may be any mechanical fastener that is configured to cooperate with a component of the absorbent article.

### Elastic Material

It is to be appreciated that components of the laminate 900 may be assembled in various ways and various combinations to create various desirable features that may differ along the lateral width and/or longitudinal length of the laminate. Such features may include, for example, Dtex values, bond patterns, aperture arrangements, elastic positioning, Average Dtex values, Average Pre-Strain values, rugosity frequencies, rugosity wavelengths, height values, and/or contact area. It is to be appreciated that differing features may be imparted to various components, such as for example, the first nonwoven substrate 202, second nonwoven substrate 204, and elastic strands 168 before and/or during stages of assembly of the laminate 900.

In some configurations, the elastic strands 168 may be disposed at a constant interval in the longitudinal direction. In other configurations, the elastic strands 168 may be disposed at different intervals in the longitudinal direction. In some configurations, the Dtex values of the elastic strands 168 may be constant or varied along the longitudinal direction. In some configurations, the elastic strands 168 in a stretched condition may be interposed and joined between uncontracted substrate layers. When the elastic strands 168 are relaxed, the elastic strands 168 returns to an unstretched condition and contracts the nonwoven substrate layers.

In some configurations, the elastic strands 168 may be parallel with each other and/or with the lateral axis 48. It is to be appreciated that the laminate 900 may be configured to include various quantities of elastic strands 168. In some configurations, elastic strands 168 may be grouped in pairs.

In some configurations, the laminate may comprise from about 1 to about 1500 elastic strands 168, or from about 10 to about 1200, or from about 50 to about 1000, or from about 100 to about 900. It is also to be appreciated that elastic strands 168 herein may comprise various Dtex values, strand spacing values, and pre-strain values. For example, in some configurations, the Average-Dtex of one or more elastic strands 168 may be greater than 500. In some configurations, the Average-Dtex of one or more elastic strands 168 may be from about 10 to about 1500, or from about 50 to about 1000, or from about 200 to about 800, specifically reciting all 1 Dtex increments within the above-recited range and all ranges formed therein or thereby. In some configurations, a plurality of elastic strands 168 may comprise an Average-Strand-Spacing of less than or equal to 4 mm. In some configurations, a plurality of elastic strands 168 may comprise an Average-Strand-Spacing from about 0.25 mm to about 4 mm, or from about 0.50 mm to about 3 mm, specifically reciting all 0.01 mm increments within the above-recited range and all ranges formed therein or thereby. In some configurations, a plurality of elastic strands 168 may comprise an Average-Strand-Spacing of greater than 4 mm. In some configurations, the Average-Pre-Strain of each of a plurality of elastic strands may be from about 50% to about 400%, specifically reciting all 1% increments within the above-recited range and all ranges formed therein or thereby. In some configurations, the elastic strands 168 may have an Average-Strand-Spacing of from about 0.25 mm to about 4 mm and an Average-Dtex of from about 10 to about 500. In some configurations, the elastic strands 168 may comprise an Average-Pre-Strain from about 75% to about 300%.

In some configurations, a first plurality of elastic strands may comprise a first Average-Pre-Strain from about 75% to about 300%, and a second plurality of elastic strands may comprise a second Average-Pre-Strain that is greater than first Average-Pre-Strain. In some configurations, a first plurality of elastic strands comprises an Average-Strand-Spacing from about 0.25 mm to about 4 mm and an Average-Dtex from about 10 to about 500; and a second plurality of elastic strands may comprise an Average-Strand-Spacing greater than about 4 mm and an Average-Dtex greater than about 450.

In some configurations, laminate 900 may have a Rugosity Frequency of from about 0.2 mm⁻¹ to about 1 mm⁻¹ and a Rugosity Wavelength of from about 0.5 mm to about 5 mm. In some configurations, the laminate 900 may comprise a Rugosity Frequency from about 0.2 mm⁻¹ to about 0.85 mm⁻¹ and Rugosity Wavelength of from about 1.2 mm to about 5 mm.

In some configurations, the elastic strands 168 may have a strand color. In some configurations, the strand color may be white. In some configurations, the strand color may be non-white. In some configurations, the first and/or second nonwoven substrate may comprise a plurality of fibers having a color that is substantially similar to the color of the elastic strands 168. Without being limited by theory, it is believed that by having elastic strands that are substantially similar to a color of the first and/or second nonwoven substrate, the elastic strands are less visually perceptible in the laminate and the laminate can appear more garment-like.

### Garment-Like Absorbent Articles

The absorbent articles of the present disclosure may have at least one component that comprises a laminate 900 as described herein. In some configurations, the laminate 900 may form a portion of the garment-facing surface 2 and/or the wearer-facing surface 4. The various absorbent article components comprising the laminate 900 may be or comprise at least a portion of a front and/or back elastic belt, a front and/or back ear, a leg cuff, an elastic waistband, or various other absorbent article components. In some configurations, the first nonwoven substrate 202 may be the front and/or back outer belt layer 64, 65 and the second nonwoven substrate may be the front and back inner belt layer 66, 67.

The absorbent article of the present disclosure may also comprise nonwoven components that comprise the nonwoven substrate 200 described herein. The various nonwoven components comprising the nonwoven substrate 200 may be or may comprise at least a portion of a backsheet, a topsheet, an outer cover, a landing zone, a wing of a sanitary napkin, or various other absorbent article nonwoven components.

In some configurations, the color scheme of the nonwoven component may be substantially similar to the color scheme of the laminate 900, forming an overall garment-like appearance to the absorbent article.

### Bio-Based Content for Components

Components of the absorbent articles described herein may at least partially be comprised of bio-based content as described in U.S. Pat. Appl. No. 2007/0219521A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and U.S. Pat. Nos. 10,166,312 and 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. No. 11,433,158, issued on Sep. 6, 2022.

### EXAMPLES

The following data and examples are provided to help illustrate the nonwoven laminates described herein. The exemplified structures are given solely for the purpose of illustration and are not to be construed as limitations of the present disclosure, as many variations thereof are possible without departing from the spirit and scope of the invention.

### Example 1

Example 1 is a stranded elastic laminate. The laminate comprises the following materials and construction.

FIRST NONWOVEN SUBSTRATE: The first nonwoven substrate is a spunbond nonwoven produced at PFNonwovens, LLC (Hazleton, PA). The total basis weight of the first nonwoven substrate is 15 gsm. The nonwoven is produced on a production line having 3 spin beams, where the fibers of the first and third beams comprise polypropylene and titanium dioxide, and the fibers of the second beam comprise polypropylene and a coral-colored masterbatch at a loading level of 2.5 percent by weight. The fibers have an average diameter ranging from 16 to 19 microns. The fibers are calender bonded using a bond pattern having S-shaped bonds and approximately 14% bond area.

SECOND NONWOVEN SUBSTRATE: The second nonwoven substrate is a 15 gsm white polypropylene nonwoven comprising titanium dioxide produced at PFNonwovens, LLC (Hazleton, PA). The fibers have an average diameter ranging from 16 to 19 microns.

The laminate is produced using the above described first nonwoven substrate on the garment-facing side and the second nonwoven substrate on the body facing side. White elastic strands (INVISTA Lycra® T837 470 DTex) are spaced at 4.1 mm and extended to 170% strain and are coated with adhesive (Bostik H2401 NF EA Omnimelt, Bostik, Wauwatosa, WI) at a weight of 18 milligrams per meter and adhered between the first and second nonwoven substrates.

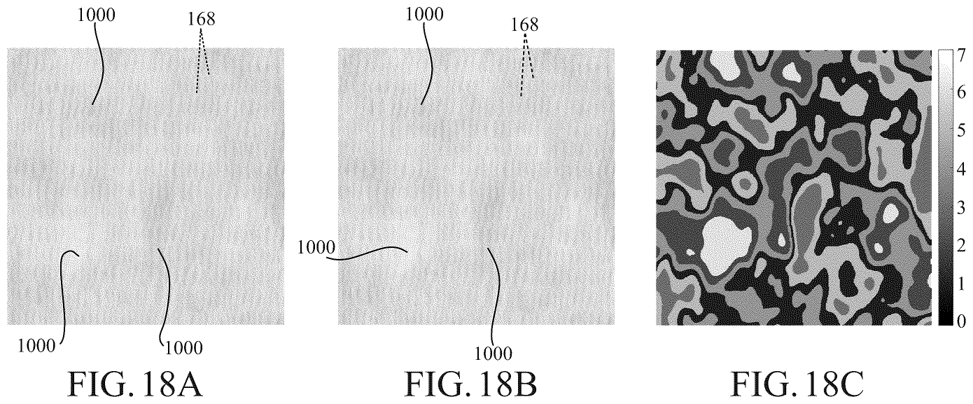

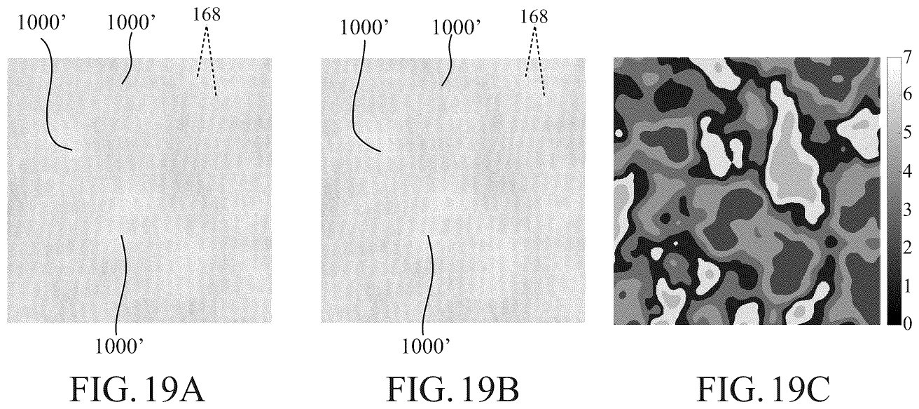

FIG. 18A is a photograph of Example 1 in a relaxed state taken from the garment-facing side showing a plurality of disruptive coloring clusters 1000. FIG. 18B is a greyscale image of the photograph of FIG. 18A. FIG. 19A is a photograph of Example 1 in an extended state taken from the garment-facing surface showing a plurality of disruptive coloring clusters 1000′. FIG. 19B is a greyscale image of the photograph of FIG. 19A.

As shown in FIGS. 18A-18B and 19A-19B, Example 1 comprises a plurality of disruptive coloring clusters 1000, 1000′ when the laminate is in both a relaxed and stretched state. It was found that the irregular shape and the non-periodic distribution of the disruptive coloring clusters can give the appearance of false edges that can help to hinder the detection and/or recognition of the elastic strands 168 in the laminate and can create a garment-like appearance.

Example 1 was analyzed using the Disruptive Coloration Test Method from the garment-facing surface. FIGS. 18C and 19C show exemplary cluster maps generated from the image of FIGS. 18A and 19A using the Disruptive Coloration Test Method described herein. Each shade of grey in the cluster map represents a color cluster according to the Disruptive Coloration Test Method. The delta E between any two pairs of disruptive coloring clusters is shown in Table 1.

TABLE 1

| | | | | | | |
|---|---|---|---|---|---|---|
| Cluster Number | 1 | 2 | 3 | 4 | 5 | 6 |

Delta E - Relaxed State

| | | | | | | |
|---|---|---|---|---|---|---|
| 1 | 0 | | | | | |
| 2 | 4 | 0 | | | | |
| 3 | 4 | 8 | 0 | | | |
| 4 | 2 | 2 | 6 | 0 | | |
| 5 | 2 | 6 | 3 | 4 | 0 | |
| 6 | 6 | 3 | 11 | 5 | 8 | 0 |

Delta E - Extended State

| | | | | | | |
|---|---|---|---|---|---|---|
| 1 | 0 | | | | | |
| 2 | 4 | 0 | | | | |
| 3 | 1 | 3 | 0 | | | |
| 4 | 3 | 2 | 1 | 0 | | |
| 5 | 4 | 8 | 5 | 6 | 0 | |
| 6 | 2 | 6 | 3 | 4 | 2 | 0 |

It was found that Example 1, which contains a nonwoven substrate comprising a first plurality of fibers comprising titanium dioxide and a second plurality of fibers comprising a coral-colored colorant, had at least one pair of disruptive coloring clusters having a delta E greater than 2 in both the relaxed and extended state. Example 1 exhibited a maximum delta E in the relaxed state of 11 and a maximum delta E in the extended state of 8. It was found that the elastic strands of Example 1 were substantially masked and the laminate had a garment or textile-like appearance. Without being limited by theory, it is believed that the masking benefit can be created when the disruptive coloring clusters have a delta E of greater than 2. It is believed that the transition between the disruptive coloring clusters can break up the outline and/or the continuity of the elastic strands in both the relaxed and extended state of the laminate.

### Example 2—Nonwoven Laminate

Example 2 is a stranded elastic laminate. The laminate comprises the following materials and construction.

FIRST NONWOVEN SUBSTRATE: The first nonwoven substrate is a spunbond nonwoven. The total basis weight of the first nonwoven substrate is 25 gsm. The fibers are bicomponent having 50% by weight polyethylene sheaths and 50% by weight polypropylene cores. The fibers have an average diameter of 16 to 18 microns. Colored masterbatches are added to the polypropylene cores. Two sublayers of fibers are calender bonded using a bond pattern having oval bonds and approximately 18% bond area to produce a unitary web. The first sublayer is approximately 10 gsm and is comprised of 2% by weight of a titanium dioxide masterbatch (Ampacet 412951, available from Ampacet Corporation, Tarrytown, NY). The second sublayer is approximately 15 gsm and is comprised of 2.5% by weight of a gray colored masterbatch (Ampacet 1202403-4, available from Ampacet Corporation).

SECOND NONWOVEN SUBSTRATE: The second nonwoven substrate is a 17 gsm white polypropylene nonwoven comprising titanium dioxide.

The laminate is produced using the above described first nonwoven substrate, with the first sublayer facing the garment-facing surface and the second sublayer facing the elastic strands, and the second nonwoven substrate on the body-facing surface. White 470 dtex INVISTA Lycra® Hyfit elastic strands are spaced at 4.1 mm and extended to 120% strain. Each of the nonwoven substrates are adhered to the strands with 9.2 gsm of meltblown glue (Bostik H2031-C5x). The glued laminate is hand cranked through a 0.050 inch MD ring-roll at a 0.050 inch depth of engagement.

Example 2 was analyzed using the Disruptive Coloration Test Method from the garment-facing surface. The delta E between any two pairs of disruptive coloring clusters is shown in Table 2.

TABLE 2

| | | | | | | |
|---|---|---|---|---|---|---|
| Cluster Number | 1 | 2 | 3 | 4 | 5 | 6 |

Delta E - Relaxed State

| | | | | | | |
|---|---|---|---|---|---|---|
| 1 | 0 | | | | | |
| 2 | 6 | 0 | | | | |
| 3 | 10 | 4 | 0 | | | |
| 4 | 3 | 3 | 7 | 0 | | |
| 5 | 3 | 9 | 13 | 6 | 0 | |
| 6 | 7 | 13 | 17 | 10 | 4 | 0 |

Delta E - Extended State

| | | | | | | |
|---|---|---|---|---|---|---|
| 1 | 0 | | | | | |
| 2 | 7 | 0 | | | | |
| 3 | 9 | 16 | 0 | | | |
| 4 | 3 | 10 | 6 | 0 | | |
| 5 | 6 | 13 | 3 | 3 | 0 | |
| 6 | 3 | 4 | 12 | 6 | 9 | 0 |

Example 2, which contains a nonwoven substrate comprising a first plurality of fibers comprising titanium dioxide and a second plurality of fibers comprising a grey-colored colorant, had at least one pair of disruptive coloring clusters with a delta E greater than 2 in both the relaxed and extended state. Example 2 exhibited a maximum delta E in a relaxed state of 17 and a maximum delta E in the extended state of 16. It was found that the elastic strands of Example 2 were substantially masked and the laminate had a garment or textile-like appearance.

### Example 3—Nonwoven Laminate

Example 3 is a stranded elastic laminate. The laminate comprises the following materials and construction.

FIRST NONWOVEN SUBSTRATE: The first nonwoven substrate is a spunbond nonwoven. The total basis weight is 20 gsm. The fibers are bicomponent having 50% by weight polyethylene sheaths and 50% by weight polypropylene cores. The fibers have an average diameter of 16 microns. Colored masterbatches are added to the polypropylene cores. Three sublayers of fibers were calender bonded using a bond pattern having oval bonds and approximately 18% bond area to produce a unitary web. The first sublayer is approximately 10 gsm and is comprised of 0.8% by weight of a yellow-colored masterbatch (Ampacet 4300524-N available from Ampacet Corporation). The second sublayer is approximately 5 gsm and is comprised of 2% by weight of teal-colored masterbatch (Ampacet LR-202088 available from Ampacet Corporation). The third sublayer is approximately 5 gsm and is comprised of 2% by weight of a pink-colored masterbatch (Ampacet LR-214290 available from Ampacet Corporation).

SECOND NONWOVEN SUBSTRATE: The above described nonwoven substrate is also used as the second nonwoven substrate.

The laminate is produced using the above described first and second nonwoven substrates, with the first nonwoven substrate oriented such that the pink colored sublayer faces the garment-facing surface and the yellow colored sublayer faces the elastic strands and the second nonwoven substrate oriented such that the pink colored sublayer faces the elastic strands and the yellow colored sublayer faces the body-facing surface. Clear 70 dtex Lycra® elastic strands are spaced at 0.8 mm and extended to 175% strain. Each of the nonwoven substrates are adhered to the strands with 9.2 gsm of meltblown glue (Bostik H2031-C5x). The glued laminate is hand cranked with a 0.075 inch pitch MD ring-roll at 0.055 inch depth of engagement.

Example 3 was analyzed using the Disruptive Coloration Test Method from the garment-facing surface. The delta E between any two pairs of disruptive coloring clusters determined via the Disruptive Coloration Test Method is shown in Table 3.

TABLE 3

| | | | | | | |
|---|---|---|---|---|---|---|
| Cluster Number | 1 | 2 | 3 | 4 | 5 | 6 |

Delta E - Relaxed State

| | | | | | | |
|---|---|---|---|---|---|---|
| 1 | 0 | | | | | |
| 2 | 6 | 0 | | | | |
| 3 | 8 | 13 | 0 | | | |
| 4 | 12 | 6 | 19 | 0 | | |
| 5 | 5 | 5 | 11 | 9 | 0 | |
| 6 | 6 | 10 | 6 | 14 | 6 | 0 |

Delta E - Extended State

| | | | | | | |
|---|---|---|---|---|---|---|
| 1 | 0 | | | | | |
| 2 | 5 | 0 | | | | |
| 3 | 11 | 11 | 0 | | | |
| 4 | 5 | 5 | 6 | 0 | | |
| 5 | 8 | 7 | 17 | 11 | 0 | |
| 6 | 6 | 8 | 6 | 4 | 13 | 0 |

Example 3, which contains a nonwoven substrate comprising a first plurality of fibers comprising a yellow-colored colorant, a second plurality of fibers comprising a teal-colored colorant, and a third plurality of fibers comprising a pink-colored colorant, had at least one pair of disruptive coloring clusters with a delta E greater than 2 in both the relaxed and extended state. Example 3 exhibited a maximum delta E in a relaxed state of 19 and a maximum delta E in the extended state of 17. It was found that the elastic strands of Example 3 were substantially masked and the laminate had a garment or textile-like appearance.

As discussed above and without wishing to be limited by theory, it is believed that nonwoven laminates comprising at least one nonwoven substrate having fibers of at least two different colors can create a disruptive coloring effect that effectively masks and/or camouflages the elastic strands of the laminate in both a relaxed and extended state, creating a garment-like appearance.

### Test Methods

### Disruptive Coloration Test Method

The Disruptive Coloration Test Method measures the color difference between disruptive coloring clusters in an elastic stranded laminate and the number of cluster intersections by the elastic strands. A flatbed scanner capable of scanning a minimum of 24-bit color at 200 dots per inch (dpi) with manual control of color management (a suitable scanner is an Epson Perfection V850 Pro from Epson America Inc., Long Beach CA, or equivalent) is used to acquire images of the elastic stranded laminate. The scanner is interfaced with a computer running color calibration software capable of calibrating the scanner against a color reflection IT8 target utilizing a corresponding reference file compliant with ANSI method IT8.7/2-1993 (suitable color calibration software is i1 Profiler available from X-Rite Grand Rapids, MI, or equivalent). The color calibration software constructs an International Color Consortium (ICC) color profile for the scanner, which is used to color correct the output images. The color corrected images are then converted into the CIE L*a*b* color space for subsequent k-means clustering, color difference analysis of the disruptive coloring clusters, and elastic strand cluster intersections using image analysis software (a suitable image color analysis software is MATLAB R2023a available from The Mathworks, Inc., Natick, MA).

### Sample Preparation:

A test sample of a nonwoven elastic stranded laminate can either be extracted from a component of an absorbent article or sampled from an unconverted nonwoven elastic stranded laminate raw material. If possible, the sample size should be large enough to image a 75 mm by 75 mm region. If the absorbent article component to be tested is smaller than this size, obtain a sample as close to these dimensions as possible.

This test is performed first with the test sample in the fully relaxed state, then the sample is extended by 70% in the direction the material is designed to elongate when in use, secured in place, and retested in the extended state. The results of this test are then reported separately for the sample in the relaxed state and in the extended state.

### Image Acquisition:

The scanner is turned on 30 minutes prior to calibration and image acquisition. Deselect any automatic color correction or color management options that may be included in the scanner software. If the automatic color management cannot be disabled, the scanner is not appropriate for this application. The recommended procedures of the color calibration software are followed to create and export an ICC color profile for the scanner. The color calibration software compares an acquired IT8 target image to a corresponding reference file to create and export the ICC color profile for the scanner, which will be applied by the scanner image acquisition software to correct the color of subsequent output images.

The scanner lid is opened, and the sample laid flat on the center of the scanner glass with the surface to be analyzed oriented toward the glass and the elastic strands oriented in the horizontal direction. The sample is then backed with a white plate and the scanner lid closed. A 75 mm by 75 mm scan containing the disruptive coloration regions to be analyzed is acquired and imported into the image analysis software at 24-bit color with a resolution of 200 dpi (approximately 7.9 pixels per mm) in reflectance mode. If the sample size is less than 75 mm by 75 mm, reduce the rectangular scanning area to the largest dimensions that can be contained within the sample. The ICC color profile is assigned to the image producing a color corrected sRGB image. This calibrated image is saved in an uncompressed format to retain the calibrated R,G,B color values, such as a TIFF file, prior to analysis.

The sRGB color calibrated image is opened in the color analysis software and converted into the CIE L*a*b* color space. This is accomplished by the following procedure. First, the sRGB data is scaled into a range of [0, 1] by dividing each of the values by 255. Then the companded sRGB channels (denoted with upper case (R,G,B), or generically V) are linearized (denoted with lower case (r,g,b), or generically v) as the following operation is performed on all three channels (R, G, and B):

$$\begin{matrix}V ∈ \{ R , G , B \} \\ v ∈ \{ r , g , b \} \\ v = \{ \begin{matrix}\frac{V}{12.92} if V \leq 0.04045 \\ {( \frac{V + 0.055}{1.055} )}^{2.4} otherwise\end{matrix} \}\end{matrix}$$

The linear r, g, and b values are then multiplied by a matrix to obtain the XYZ Tristimulus values according to the following formula:

$$[ \begin{matrix}X \\ Y \\ Z\end{matrix} ] = [ \begin{matrix}0.4124 & 0.3576 & 0.1805 \\ 0.2126 & 0.7152 & 0.0722 \\ 0.0193 & 0.1192 & 0.9505\end{matrix} ] [ \begin{matrix}r \\ g \\ b\end{matrix} ]$$

The XYZ Tristimulus values are rescaled by multiplying the values by 100, and then converted into CIE 1976 L*a*b* values as defined in CIE 15:2004 section 8.2.1.1 using D65 reference white.

Using the image analysis software, the CIE L*a*b* image is blurred using a Gaussian filter with a sigma value of 10 to remove small scale color variations allowing for analysis of the larger scale disruptive coloration in the sample. Next, perform k-means clustering on the image using default settings in addition to the following specifications: Number of clusters=6, maximum number of iterations=1000; and the number of replicates=5. The k-means clustering method is an unsupervised machine learning technique used to identify clusters, or groups of pixels in an image, whose L*, a*, and b* color values are more similar to other pixels in their cluster than they are to pixels in other clusters. The image analysis software generates a cluster map in which it assigns a cluster number from 1 to 6 to each of the pixels in the image based on the disruptive coloring cluster that each pixel was assigned to (e.g., FIGS. 18C and 19C).

### Color Cluster ΔE Measurement:

The average L*, a*, and b* values within each of the identified disruptive coloring clusters is calculated. The Delta E (ΔE) color difference is calculated between each cluster and all the other clusters. To do this identify the average L*, a*, and b* values from a first cluster as L*₁, a*₁, and b*₁, and identify the average L*, a*, and b* values from a second cluster as L*₂, a*₂, and b*₂. The Delta E value is then calculated according to the following equation:

$$Delta E = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

This procedure is repeated for all possible combinations of cluster pairs from the 6 different disruptive coloring clusters, with the ΔE for each recorded to the nearest whole number in a Delta E matrix as exemplified in Tables 1 through 3.

Additionally, identify and record the maximum Delta E value from the matrix for the sample analysis in both the relaxed and extended states to the nearest whole number.

### Color Cluster Strand Intersection Measurement:

Identify an elastic strand in the sRGB color calibrated image and, using the image analysis software, draw a line tracing the identified strand across the entire image. Transfer the drawn line to the cluster map and count the total number of times the line transitions from one cluster to another one and report this value as the number of times the elastic strand intersects a disruptive coloring cluster to the nearest whole number.

Additionally, extract out the list of unique cluster numbers that are intersected by the identified elastic strand and use the Delta E matrix for that image to identify the Delta E values for all possible combinations of disruptive coloring cluster pairs intersected by the elastic strand.

### Average Decitex (Average-Dtex)

The Average Decitex Method is used to calculate the Average-Dtex on a length-weighted basis for elastic fibers present in an entire article, or in a specimen of interest extracted from an article. The decitex value is the mass in grams of a fiber present in 10,000 meters of that material in the relaxed state. The decitex value of elastic fibers or elastic laminates containing elastic fibers is often reported by manufacturers as part of a specification for an elastic fiber or an elastic laminate including elastic fibers. The Average-Dtex is to be calculated from these specifications if available. Alternatively, if these specified values are not known, the decitex value of an individual elastic fiber is measured by determining the cross-sectional area of a fiber in a relaxed state via a suitable microscopy technique such as scanning electron microscopy (SEM), determining the composition of the fiber via Fourier Transform Infrared (FT-IR) spectroscopy, and then using a literature value for density of the composition to calculate the mass in grams of the fiber present in 10,000 meters of the fiber. The manufacturer-provided or experimentally measured decitex values for the individual elastic fibers removed from an entire article, or specimen extracted from an article, are used in the expression below in which the length-weighted average of decitex value among elastic fibers present is determined.

The lengths of elastic fibers present in an article or specimen extracted from an article is calculated from overall dimensions of and the elastic fiber pre-strain ratio associated with components of the article with these or the specimen, respectively, if known. Alternatively, dimensions and/or elastic fiber pre-strain ratios are not known, an absorbent article or specimen extracted from an absorbent article is disassembled and all elastic fibers are removed. This disassembly can be done, for example, with gentle heating to soften adhesives, with a cryogenic spray (e.g., Quick-Freeze, Miller-Stephenson Company, Danbury, CT), or with an appropriate solvent that will remove adhesive but not swell, alter, or destroy elastic fibers. The length of each elastic fiber in its relaxed state is measured and recorded in millimeters (mm) to the nearest mm.

### Calculation of Average-Dtex

For each of the individual elastic fibers fᵢ of relaxed length Lᵢ and fiber decitex value dᵢ (obtained either from the manufacturer's specifications or measured experimentally) present in an absorbent article, or specimen extracted from an absorbent article, the Average-Dtex for that absorbent article or specimen extracted from an absorbent article is defined as:

$$Average - Dtex = \frac{{\sum }_{i = 1}^{n} ( {L}_{i} \times {d}_{i} )}{{\sum }_{i = 1}^{n} {L}_{i}}$$

where n is the total number of elastic fibers present in an absorbent article or specimen extracted from an absorbent article. The Average-Dtex is reported to the nearest integer value of decitex (grams per 10 000 m).

If the decitex value of any individual fiber is not known from specifications, it is experimentally determined as described below, and the resulting fiber decitex value(s) are used in the above equation to determine Average-Dtex.

### Experimental Determination of Decitex Value for a Fiber

For each of the elastic fibers removed from an absorbent article or specimen extracted from an absorbent article according to the procedure described above, the length of each elastic fiber Lₖ in its relaxed state is measured and recorded in millimeters (mm) to the nearest mm. Each elastic fiber is analyzed via FT-IR spectroscopy to determine its composition, and its density ρₖ is determined from available literature values. Finally, each fiber is analyzed via SEM. The fiber is cut in three approximately equal locations perpendicularly along its length with a sharp blade to create a clean cross-section for SEM analysis. Three fiber segments with these cross sections exposed are mounted on an SEM sample holder in a relaxed state, sputter coated with gold, introduced into an SEM for analysis, and imaged at a resolution sufficient to clearly elucidate fiber cross sections. Fiber cross sections are oriented as perpendicular as possible to the detector to minimize any oblique distortion in the measured cross sections. Fiber cross sections may vary in shape, and some fibers may consist of a plurality of individual filaments. Regardless, the area of each of the three fiber cross sections is determined (for example, using diameters for round fibers, major and minor axes for elliptical fibers, and image analysis for more complicated shapes), and the average of the three areas aₖ for the elastic fiber, in units of micrometers squared (μm²), is recorded to the nearest 0.1 μm². The decitex dₖ of the kth elastic fiber measured is calculated by:

$${d}_{k} = 1 \; 0000 m \times {a}_{k} \times {ρ}_{k} \times 1 {0}^{- 6}$$

where dₖ is in units of grams (per calculated 10,000 meter length), aₖ is in units of μm², and ρₖ is in units of grams per cubic centimeter (g/cm³). For any elastic fiber analyzed, the experimentally determined Lₖ and dₖ values are subsequently used in the expression above for Average-Dtex.

### Average-Strand-Spacing

Using a ruler calibrated against a certified NIST ruler and accurate to 0.5 mm, measure the distance between the two distal strands within a section to the nearest 0.5 mm, and then divide by the number of strands in that section−1

$$Average - Strand - Spacing = d / ( n - 1 ) where n > 1$$

report to the nearest 0.1 mm.

### Average-Pre-Strain

The Average-Pre-Strain of a specimen are measured on a constant rate of extension tensile tester (a suitable instrument is the MTS Insight using Testworks 4.0 Software, as available from MTS Systems Corp., Eden Prairie, MN) using a load cell for which the forces measured are within 1% to 90% of the limit of the cell. Articles are conditioned at 23° C.±2 C.° and 50%±2% relative humidity for 2 hours prior to analysis and then tested under the same environmental conditions.

Program the tensile tester to perform an elongation to break after an initial gage length adjustment. First raise the cross head at 10 mm/min up to a force of 0.05N. Set the current gage to the adjusted gage length. Raise the crosshead at a rate of 100 mm/min until the specimen breaks (force drops 20% after maximum peak force). Return the cross head to its original position. Force and extension data is acquired at a rate of 100 Hz throughout the experiment.

Set the nominal gage length to 40 mm using a calibrated caliper block and zero the crosshead. Insert the specimen into the upper grip such that the middle of the test strip is positioned 20 mm below the grip. The specimen may be folded perpendicular to the pull axis, and placed in the grip to achieve this position. After the grip is closed the excess material can be trimmed. Insert the specimen into the lower grips and close. Once again, the strip can be folded, and then trimmed after the grip is closed. Zero the load cell. The specimen should have a minimal slack but less than 0.05 N of force on the load cell. Start the test program.

From the data construct a Force (N) verses Extension (mm). The Average-Pre-Strain is calculated from the bend in the curve corresponding to the extension at which the nonwovens in the elastic are engaged. Plot two lines, corresponding to the region of the curve before the bend (primarily the elastics), and the region after the bend (primarily the nonwovens). Read the extension at which these two lines intersect, and calculate the % Pre-Strain from the extension and the corrected gage length. Record as % Pre-strain 0.1%. Calculate the arithmetic mean of three replicate samples for each elastomeric laminate and Average-Pre-Strain to the nearest 0.1%.

### Combinations

A1. An absorbent article comprising:
a garment-facing surface;
a wearer-facing surface;
a topsheet;
a backsheet;
an absorbent core positioned at least partially intermediate the topsheet and the backsheet; and
a component comprising a laminate, wherein the laminate comprises a first nonwoven substrate, a second nonwoven substrate, and a plurality of elastic strands positioned at least partially intermediate the first nonwoven substrate and the second nonwoven substrate;
wherein the first nonwoven substrate comprises a first plurality of fibers having a first color and a second plurality of fibers having a second color, wherein the first color and the second color are different;
wherein the laminate comprises a first side and a second side opposite the first side, wherein the first nonwoven substrate forms a portion of the first side;
wherein the first side of the laminate comprises a plurality of disruptive coloring clusters.
   A2. The absorbent article of Paragraph A1, wherein at least a pair of disruptive coloring clusters have a delta E of greater than 2, or from 2 to 100, as measured according to the Disruptive Coloration Test Method in a relaxed state.
   A3. The absorbent article of Paragraph A1 or A2, wherein at least a pair of disruptive coloring clusters have a delta E of greater than 2, or from 2 to 100, as measured according to the Disruptive Coloration Test Method in an extended state.
   A4. The absorbent article of Paragraph A1, wherein the disruptive coloring clusters have a first maximum delta E in a relaxed state and a second maximum delta E in an extended state, as measured according to the Disruptive Coloration Test Method, wherein the first maximum delta E is greater than the second maximum delta E.
   A5. The absorbent article of Paragraph A1, wherein the plurality of elastic strands intersect at least three of the disruptive coloring clusters in the extended state, as measured according to the Disruptive Coloration Test Method.
   A6. The absorbent article of Paragraph A5, wherein at least a pair of the disruptive coloring clusters that have been intersected by an elastic strand have a delta E of greater than 2, as measured according to the Disruptive Coloration Test Method A7. The absorbent article of any of Paragraphs A1 to A6, wherein the first color is white and the second color is a non-white color.
   A8. The absorbent article of any of Paragraphs A1 to A6, wherein the first color and the second color are non-white colors.
   A9. The absorbent article of any of Paragraphs A1 to A8, wherein at least one of the first plurality of fibers and the second plurality of fibers comprise a colorant.
   A10. The absorbent article of any of Paragraphs A1 to A8, wherein the first plurality of fibers comprise a colorant and the second plurality of fibers does not comprise a colorant.
   A11. The absorbent article of any of Paragraphs A1 to A10, wherein the first side of the laminate forms a portion of the garment-facing surface.
   A12. The absorbent article of any of Paragraphs A1 to A11, wherein the first nonwoven substrate comprises a third plurality of fibers having a third color that is different from the first color and the second color.
   A13. The absorbent article of any of Paragraphs A1 to A12, wherein the second nonwoven substrate comprises at least one of the first plurality of fibers and the second plurality of fibers.
   A14. The absorbent article of any of Paragraphs A1 to A13, wherein at least one of the first plurality of fibers and the second plurality of fibers are spunbond.
   A15. The absorbent article of any of Paragraphs A1 to A14, wherein the first nonwoven substrate comprises from about 15% to about 85% of the first plurality of fibers.
   A16. The absorbent article of any of Paragraphs A1 to A15, wherein the first nonwoven substrate comprises from about 15% to about 85% of the second plurality of fibers.
   A17. The absorbent article of any of Paragraphs A1 to A16, wherein the first and second plurality of fibers are substantially oriented in the machine direction.
   A18. The absorbent article of any of Paragraphs A1 to A17, wherein the second nonwoven substrate forms a portion of the wearer-facing surface, wherein the second nonwoven substrate has a color that is substantially similar to a color of the plurality of elastic strands.
   A19. The absorbent article of any of Paragraphs A1 to A18, wherein the plurality of disruptive coloring clusters have an irregular shape.
   A20. The absorbent article of any of Paragraphs A1 to A19, wherein the plurality of disruptive coloring clusters are positioned in a non-periodic pattern.
   A21. The absorbent article of any of Paragraphs A1 to A20, wherein a delta E between the first color and the second color is greater than 5, preferably from 5 to 300.
   A22. The absorbent article of any of Paragraphs A1 to A21, wherein the component is selected from one or more of:
(i) an elastic belt;
(ii) a back ear;
(iii) an elastic waistband; and
(iv) a leg cuff.
   B1. An absorbent article comprising: a garment-facing surface; a wearer-facing surface; a topsheet; a backsheet; an absorbent core positioned between the topsheet and the backsheet; and an elastic belt connected with the backsheet, the elastic belt comprising a laminate, wherein the laminate comprises a first nonwoven substrate, a second nonwoven substrate, and a plurality of elastic strands positioned between the first nonwoven substrate and the second nonwoven substrate; the first nonwoven substrate consisting of first fibers having a first color; the second nonwoven substrate consisting of second fibers having a second color, wherein the first color and the second color are different; wherein the laminate comprises a first side and a second side opposite the first side, wherein the first nonwoven substrate forms a portion of the first side; wherein the portion of the first side of the laminate comprises a plurality of disruptive coloring clusters.
   B2. The absorbent article of paragraph B1, wherein at least a pair of the disruptive coloring clusters have a delta E of greater than 2, as measured according to the Disruptive Coloration Test Method in a relaxed state.
   B3. The absorbent article of paragraph B2, wherein the disruptive coloring clusters comprise a maximum delta E of 12 or less.
   B4. The absorbent article of any of paragraphs B1 to B3, wherein at least one of the first plurality of fibers and the second plurality of fibers comprise a colorant.
   B5. The absorbent article of any of paragraphs B1 to B3, wherein the first plurality of fibers comprise a colorant and the second plurality of fibers does not comprise a colorant.
   B6. The absorbent article of any of paragraphs B1 to B5, wherein the first side of the laminate forms a portion of the garment-facing surface.
   B7. An absorbent article comprising: a garment-facing surface; a wearer-facing surface; a topsheet; a backsheet; an absorbent core positioned between the topsheet and the backsheet; and an elastic belt connected with the backsheet; the backsheet comprising a laminate, wherein the laminate comprises a nonwoven substrate and a film substrate; the nonwoven substrate consisting of first fibers having a first color; the film substrate consisting of material having a second color, wherein the first color and the second color are different; wherein the laminate comprises a first side and a second side opposite the first side, wherein the nonwoven substrate forms a portion of the first side; wherein the portion of the first side of the laminate comprises a plurality of disruptive coloring clusters.
   B8. The absorbent article of paragraph B7, wherein at least a pair of the disruptive coloring clusters have a delta E of greater than 2, as measured according to the Disruptive Coloration Test Method.
   B9. The absorbent article of paragraph B8, wherein the disruptive coloring clusters comprise a maximum delta E of 15 or less.
   B10. The absorbent article of any of paragraphs B7 to B9, wherein at least one of the first plurality of fibers and the second plurality of fibers comprise a colorant.
   B11. The absorbent article of any of paragraphs B7 to B10, wherein the first fibers comprise a colorant.
   B12. The absorbent article of any of paragraphs B7 to B11, wherein the first side of the laminate forms a portion of the garment-facing surface.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All ranges disclosed herein specifically recite all 0.1 increments within the specified ranges and all ranges formed therein or thereby.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. An absorbent article comprising:
a garment-facing surface;
a wearer-facing surface;
a topsheet;
a backsheet;
an absorbent core positioned at least partially intermediate the topsheet and the backsheet; and
a component comprising a laminate, wherein the laminate comprises a first nonwoven substrate, a second nonwoven substrate, and a plurality of elastic strands positioned at least partially intermediate the first nonwoven substrate and the second nonwoven substrate;
wherein the first nonwoven substrate comprises a first plurality of fibers having a first color and a second plurality of fibers having a second color, wherein the first color and the second color are different;
wherein the laminate comprises a first side and a second side opposite the first side, wherein the first nonwoven substrate forms a portion of the first side;
wherein the first side of the laminate comprises a plurality of disruptive coloring clusters.

2. The absorbent article of claim 1, wherein at least a pair of the disruptive coloring clusters have a delta E of greater than 2, as measured according to the Disruptive Coloration Test Method in a relaxed state.

3. The absorbent article of claim 2, wherein at least a pair of the disruptive coloring clusters have a delta E of greater than 2, as measured according to the Disruptive Coloration Test Method in an extended state.

4. The absorbent article of claim 1, wherein the first color is white and the second color is a non-white color.

5. The absorbent article of claim 1, wherein the first color and the second color are non-white colors.

6. The absorbent article of claim 1, wherein at least one of the first plurality of fibers and the second plurality of fibers comprise a colorant.

7. The absorbent article of claim 1, wherein the first plurality of fibers comprise a colorant and the second plurality of fibers does not comprise a colorant.

8. The absorbent article of claim 1, wherein the first side of the laminate forms a portion of the garment-facing surface.

9. The absorbent article of claim 1, wherein the first nonwoven substrate comprises a third plurality of fibers having a third color that is different from the first color and the second color.

10. The absorbent article of claim 1, wherein the second nonwoven substrate comprises at least one of the first plurality of fibers and the second plurality of fibers.

11. The absorbent article of claim 1, wherein the component is selected from one or more of:
(i) an elastic belt;
(ii) a back ear;
(iii) an elastic waistband; and
(iv) a leg cuff.

12. An absorbent article comprising:
a topsheet;
a backsheet;
an absorbent core positioned at least partially intermediate the topsheet and the backsheet; and
a component comprising a laminate, wherein the laminate comprises a first nonwoven substrate, a second nonwoven substrate, and a plurality of elastic strands positioned at least partially intermediate the first nonwoven substrate and the second nonwoven substrate;
wherein the first nonwoven substrate comprises a first plurality of fibers having a first color and a second plurality of fibers having a second color, wherein the first color and the second color are different;
wherein the laminate comprises a first side and a second side opposite the first side, wherein the first nonwoven substrate forms a portion of the first side;
wherein the first side of the laminate comprises a plurality of disruptive coloring clusters,
wherein the disruptive coloring clusters have a first maximum delta E in a relaxed state and a second maximum delta E in an extended state, as measured according to the Disruptive Coloration Test Method, wherein the first maximum delta E is greater than the second maximum delta E.

13. The absorbent article of claim 12, wherein the first nonwoven substrate comprises from about 15% to about 85% of the first plurality of fibers.

14. The absorbent article of claim 13, wherein the first nonwoven substrate comprises from about 15% to about 85% of the second plurality of fibers.

15. The absorbent article of claim 12, wherein the first and second plurality of fibers are substantially oriented in the machine direction.

16. An absorbent article comprising:
a garment-facing surface;
a wearer-facing surface;
a topsheet;
a backsheet;
an absorbent core positioned at least partially intermediate the topsheet and the backsheet; and
a component comprising a laminate, wherein the laminate comprises a first nonwoven substrate, a second nonwoven substrate, and a plurality of elastic strands positioned at least partially intermediate the first nonwoven substrate and the second nonwoven substrate;
wherein the first nonwoven substrate comprises a first plurality of fibers having a first color and a second plurality of fibers having a second color, wherein the first color and the second color are different;
wherein the laminate comprises a first side and a second side opposite the first side, wherein the first nonwoven substrate forms a portion of the first side;
wherein the first side of the laminate comprises a plurality of disruptive coloring clusters;
wherein the plurality of elastic strands intersect at least three of the disruptive coloring clusters in the extended state, as measured according to the Disruptive Coloration Test Method.

17. The absorbent article of claim 16, wherein the first nonwoven substrate forms a portion of the garment-facing surface.

18. The absorbent article of claim 17, wherein the second nonwoven substrate forms a portion of the wearer-facing surface, wherein the second nonwoven substrate has a color that is substantially similar to a color of the plurality of elastic strands.

19. The absorbent article of claim 16, wherein the plurality of disruptive coloring clusters have an irregular shape.

20. The absorbent article of claim 19, wherein the plurality of disruptive coloring clusters are positioned in a non-periodic pattern.
